(19) United States
(12) Patent Application Publication
(10) Pub. No.: US 2025/0216329 A1
(43) Pub. Date: Jul. 3, 2025

(54) DRUG DETECTION

(71) Applicant: THE UNIVERSITY OF BATH, BATH (GB)

(72) Inventors: Christopher R PUDNEY, Bath (GB); Rachael ANDREWS, Bath (GB); Gyles COZIER, Bath (GB); Matthew GARDNER, Bath (GB)

(21) Appl. No.: 18/403,424

(22) Filed: Jan. 3, 2024

(51) Int. Cl. G01N 21/64; G01N 33/94; G16C 20/20

(52) U.S. Cl. CPC G01N 21/6428; G01N 33/948; G01N 33/9486; G16C 20/20; G01N 2021/6419

(57) ABSTRACT

Aspects and embodiments relate to apparatus and methods to identify whether a drug is present in a provided sample. One aspect provides a drug detection method to identify whether a drug is present in a provided sample, the method comprising: determining a first fluorescence spectral matrix associated with the provided sample by: excitation of the provided sample at a first excitation wavelength; and receiving a fluorescence emission spectrum generated by the provided sample at the first excitation wavelength; repeating the excitation and receiving steps across a range of excitation wavelengths which differ from the first excitation wavelength; determining a wavelength for irradiation of the sample to induce a photochemical change in the provided sample and irradiating the provided sample with radiation having the determined wavelength; determining a second fluorescence spectral matrix associated with the provided sample by: excitation of the provided sample at a first excitation wavelength; and receiving a fluorescence emission spectrum generated by the provided sample at the first excitation wavelength; repeating the excitation and receiving steps across a range of excitation wavelengths which differ from the first excitation wavelength; comparing the determined first and second fluorescence spectral matrix associated with the provided sample with a library of first and second fluorescence spectral matrices obtained from a plurality of drug samples; and if a match within a predetermined threshold is revealed by said comparison, triggering a positive identification of a drug in the provided sample. Aspects recognise that the rapid detection of certain drugs can be enhanced by augmenting a fluorescence spectral fingerprint based detection methodology with photochemical reactivity tracking. The hybrid photochemical fingerprinting methodology in accordance with aspects allows for capture of information on both: new fluorescent species that emerge as a result of a specifically induced photochemical reaction(s) notable detectable drugs, but also the loss of fluorescent/absorptive species as they are photochemically degraded. Both such physical changes are reflective of the specific chemistry of the analyte (drug) which is being tested for.

### TECHNOLOGICAL FIELD

Various example embodiments relate to apparatus and methods to identify whether a drug is present in a provided sample.

### BACKGROUND

Illicit substance use, for example, illicit use of various controlled substances, drugs and/or medications, is prevalent across Europe and the rest of the world. It is imperative that drug detection methods adapt to meet the challenge of increased substance use and abuse.

Various substance detection methods are known. For example, a variety of drug testing methods are available including: screening, colorimetric detection, immunochemical assays, and chromatographic methods. Whilst there are advantages and disadvantages to each method, colorimetric detection is typically favoured in a point of care setting due to being both rapid and portable. However, colorimetric detection tends to be specific to individual structures. In contrast, chromatographic methods including Liquid Chromatography Mass Spectroscopy (LC-MS) and Mass Spectroscopy (MS) provide a more advanced detection method capable of resolving a large range of compounds, with a low limit of detection. However, the high associated costs and lack of portability renders chromatographic methods generally unsuitable for mobile drug testing.

By way of one specific example, synthetic cannabinoid receptor agonist (SCRA) use is prevalent across Europe and the United States. New, structurally advanced, generations of the drug are emerging, so it is imperative that drug detection methods advance at the same rate. SCRAs are a chemically diverse and evolving group, which makes rapid detection challenging. It has been shown that fluorescence spectral fingerprinting (FSF) has potential to provide a rapid assessment of SCRA presence both directly from street material with minimal processing and in subject saliva samples.

It may be desirable to enhance sensitivity and/or discriminatory ability of SCRA detection approaches, for example, to accelerate delivery of a point-of-care technology which can be used confidently by a range of stakeholders; from medical to prison staff.

### BRIEF SUMMARY

The scope of protection sought for various example embodiments of the invention is set out in the independent claims. The example embodiments and features, if any, described in this specification that do not fall under the scope of the independent claims are to be interpreted as examples useful for understanding various embodiments of the invention.

According to various, but not necessarily all, example embodiments there is provided: a drug detection method to identify whether a drug is present in a provided sample, the method comprising: determining a first fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
determining a wavelength for irradiation of the provided sample to induce a photochemical change in the provided sample and irradiating the provided sample with radiation having the determined wavelength;
determining a second fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
comparing the determined first and second fluorescence spectral matrix associated with the provided sample with a library of first and second fluorescence spectral matrices obtained from a plurality of drug samples; and
if a match within a predetermined threshold is revealed by said comparison, triggering a positive identification of a drug in the provided sample.

Described aspects recognise that the rapid detection of certain drugs can be enhanced by augmenting a fluorescence spectral fingerprint based detection methodology with photochemical reactivity tracking. The hybrid photochemical fingerprinting methodology in accordance with aspects allows for capture of information on both: new fluorescent species that emerge as a result of a specifically induced photochemical reaction(s) notable detectable drugs, but also the loss of fluorescent/absorptive species as they are photochemically degraded. Both such physical changes are reflective of the specific chemistry of the analyte (drug) which is being tested for.

According to some embodiments, determining a wavelength for irradiation of the provided sample to induce a photochemical change in the provided sample comprises: determining a wavelength of an absorption maxima of the provided sample and irradiating the provided sample with irradiation having a wavelength close to the determined absorption maxima to induce a photochemical change in the provided sample.

According to some embodiments, determining a wavelength for irradiation of the provided sample to induce a photochemical change in the provided sample comprises: measuring an absorption spectrum of a plurality of drugs and evaluating the measured absorption spectra to select one or more wavelength for irradiation of the provided sample.

According to some embodiments, the wavelength for irradiation comprises a wavelength in the ultraviolet region of the spectrum.

According to some embodiments, the wavelength for irradiation of the provided sample may comprise a wavelength, or plurality of wavelengths, in the range from around 230 nm to around 400 nm. According to some embodiments, the wavelength for irradiation may comprise a range of wavelengths. The rage of wavelengths may comprise, for example, a range of UV wavelengths. Evaluation of irradiation wavelength may comprise selecting a range of UV wavelengths with which to irradiate a sample, that irradiation may be performed in a non-provided-sample-specific manner, such that a provided sample is irradiated, for example, with broadband UV, or a cycle of different UV wavelengths.

According to some embodiments, the drug comprises at least one of the following: a photochemically active aromatic molecule; or a molecule which photochemically changes to form an aromatic molecule; or a synthetic cannabinoid receptor agonist; or a cannabinoid; or an opioid; or a benzodiazepine; or a cathinone; or a steroid or a sedative.

According to some embodiments, the provided sample comprises: a drug in solution, and optionally the provided sample comprises at least one of the following: a drug in ethanol solution; or a drug in saliva; or a combusted drug in saliva; or a drug in solid form.

According to some embodiments, the provided sample comprises: a drug adsorbed onto a physical matrix, and optionally wherein the physical matrix comprises paper.

According to some embodiments, triggering a positive identification of a drug comprises identifying which of the plurality of drug samples in the library is a source of a first and second fluorescence spectral matrix which both match said determined first and second fluorescence spectral matrix associated with the provided sample within the threshold.

According to some embodiments, triggering a positive identification of a drug comprises identifying which of the plurality of drug samples in the library is a source of a difference map between a first and second fluorescence spectral matrix which matches a difference map created by comparison of the determined first and second fluorescence spectral matrix associated with the provided sample within the threshold.

According to some embodiments, triggering a positive identification of a drug comprises identifying which of the plurality of drug samples in the library is a source of a difference map between a first and second fluorescence spectral matrix which matches a difference map created by comparison of the determined first and second fluorescence spectral matrix associated with the provided sample, and a first fluorescence spectral matrix which matches a determined first fluorescence spectral matrix associated with the provided sample within the threshold.

According to some embodiments, irradiating the provided sample comprises irradiating the provided sample for a predetermined time period at a selected intensity and optionally wherein the predetermined time period and intensity are selected to be likely to induce a photochemical change in a drug in the provided sample.

According to some, but not necessarily all, aspects there is provided a drug detection apparatus configured to identify whether a drug is present in a provided sample, the apparatus comprising: a controller, an excitation source and an emission detector;
the controller comprising at least one processor; and at least one memory storing instructions that, when executed by the at least one processor, cause the controller at least to perform:
determining a first fluorescence spectral matrix associated with the provided sample by:
causing excitation of the provided sample by the excitation source at a first excitation wavelength; and
receiving, from the emission detector, a fluorescence emission spectrum generated by the provided sample at the first excitation wavelength;
repeating the excitation and receiving steps across a range of excitation wavelengths which differ from the first excitation wavelength;
storing excitation and emission data obtained in each excitation and receiving step; and
communicating with the stored data to create the first fluorescence spectral matrix associated with the provided sample;
determining a wavelength for irradiation of the provided sample to induce a photochemical change in the provided sample and irradiating the provided sample with radiation having the determined wavelength;
determining a second fluorescence spectral matrix associated with the provided sample by:
causing excitation of the provided sample by the excitation source at a first excitation wavelength; and
receiving, from the emission detector, a fluorescence emission spectrum generated by the provided sample at the first excitation wavelength;
repeating the excitation and receiving steps across a range of excitation wavelengths which differ from the first excitation wavelength;
storing excitation and emission data obtained in each excitation and receiving step; and
communicating with the stored data to create the second fluorescence spectral matrix associated with the provided sample;
comparing the determined first and second fluorescence spectral matrix associated with the provided sample with a library of first and second fluorescence spectral matrices obtained from a plurality of drug samples; and
if a match within a predetermined threshold is revealed by said comparison, triggering a positive identification of a drug in the provided sample.

According to some embodiments, determining a wavelength for irradiation of the provided sample to induce a photochemical change in the provided sample comprises: determining a wavelength of an absorption maxima of the provided sample and irradiating the provided sample with irradiation having a wavelength close to the determined absorption maxima to induce a photochemical change in the provided sample.

According to some embodiments, determining a wavelength for irradiation of the provided sample to induce a photochemical change in the provided sample comprises: measuring an absorption spectrum of a plurality of drugs and evaluating the measured absorption spectra to select one or more wavelength for irradiation of the provided sample.

According to some embodiments, the wavelength for irradiation comprises a wavelength in the ultraviolet region of the spectrum.

According to some embodiments, the wavelength for irradiation of the provided sample may comprise a wavelength, or plurality of wavelengths, in the range from around 230 nm to around 400 nm. According to some embodiments, the wavelength for irradiation may comprise a range of wavelengths. The rage of wavelengths may comprise, for example, a range of UV wavelengths. Evaluation of irradiation wavelength may comprise selecting a range of UV wavelengths with which to irradiate a sample, that irradiation may be performed in a non-provided-sample-specific manner, such that a provided sample is irradiated, for example, with broadband UV, or a cycle of different UV wavelengths.

According to some embodiments, the drug comprises at least one of the following: a photochemically active aromatic molecule; or a molecule which photochemically changes to form an aromatic molecule; or a synthetic cannabinoid receptor agonist; or a cannabinoid; or an opioid; or a benzodiazepine; or a cathinone; or a steroid or a sedative.

According to some embodiments, the provided sample comprises: a drug in solution, and optionally the provided sample comprises at least one of the following: a drug in ethanol solution; or a drug in saliva; or a combusted drug in saliva; or a drug in solid form.

According to some embodiments, the provided sample comprises: a drug adsorbed onto a physical matrix, and optionally wherein the physical matrix comprises paper.

According to some embodiments, triggering a positive identification of a drug comprises identifying which of the plurality of drug samples in the library is a source of a first and second fluorescence spectral matrix which both match said determined first and second fluorescence spectral matrix associated with the provided sample within the threshold.

According to some embodiments, triggering a positive identification of a drug comprises identifying which of the plurality of drug samples in the library is a source of a difference map between a first and second fluorescence spectral matrix which matches a difference map created by comparison of the determined first and second fluorescence spectral matrix associated with the provided sample within the threshold.

According to some embodiments, triggering a positive identification of a drug comprises identifying which of the plurality of drug samples in the library is a source of a difference map between a first and second fluorescence spectral matrix which matches a difference map created by comparison of the determined first and second fluorescence spectral matrix associated with the provided sample, and a first fluorescence spectral matrix which matches a determined first fluorescence spectral matrix associated with the provided sample within the threshold.

According to some embodiments, irradiating the provided sample comprises irradiating the provided sample for a predetermined time period at a selected intensity and optionally wherein the predetermined time period and intensity are selected to be likely to induce a photochemical change in a drug in the provided sample.

According to some embodiments, the excitation source comprises: an array of different LEDs selected to have an emission spectrum which, when taken together, spans a wavelength range from 250 nm to 400 nm.

According to some embodiments, the controller is configured to control said array of different LEDs to selectively switch one or more LEDs in said array on or off and thereby control said first wavelength.

According to some embodiments, the emission detector comprises a monochromated spectrometer.

According to some embodiments, the library is stored on locally provided memory; and optionally wherein the apparatus comprises communication circuitry configured to communicate with a remotely stored library.

According to some, but not necessarily all, aspects, there is provided an apparatus for drug detection configured to identify whether a drug is present in a provided sample, the apparatus comprising:
circuitry configured to perform determining a first fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
circuitry configured to perform determining a wavelength for irradiation of the provided sample to induce a photochemical change in the provided sample and irradiating the provided sample with radiation having the determined wavelength;
circuitry configured to perform determining a second fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
circuitry configured to perform comparing the determined first and second fluorescence spectral matrix associated with the provided sample with a library of first and second fluorescence spectral matrices obtained from a plurality of drug samples; and
if a match within a predetermined threshold is revealed by said comparison, circuitry configured to perform triggering a positive identification of a drug in the provided sample.

The circuitry may be configured perform the optional features set out in relation to the apparatus mentioned above.

According to some, but not necessarily all, aspects, there is provided a non-transitory computer readable medium comprising program instructions stored thereon for performing a drug detection method to identify whether a drug is present in a provided sample, the instructions comprising at least the following:
determining a first fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
determining a wavelength for irradiation of the provided sample to induce a photochemical change in the provided sample and irradiating the provided sample with radiation having the determined wavelength;
determining a second fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
comparing the determined first and second fluorescence spectral matrix associated with the provided sample with a library of first and second fluorescence spectral matrices obtained from a plurality of drug samples; and
if a match within a predetermined threshold is revealed by said comparison, triggering a positive identification of a drug in the provided sample.

The instructions may be for performing the optional features set out in relation to the method mentioned above.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION

The patent or application file contains at least one drawing executed in color. Copies of this patent or patent application publication with color drawing(s) will be provided by the Office upon request and payment of the necessary fee.

Some example embodiments will now be described with reference to the accompanying drawings in which:

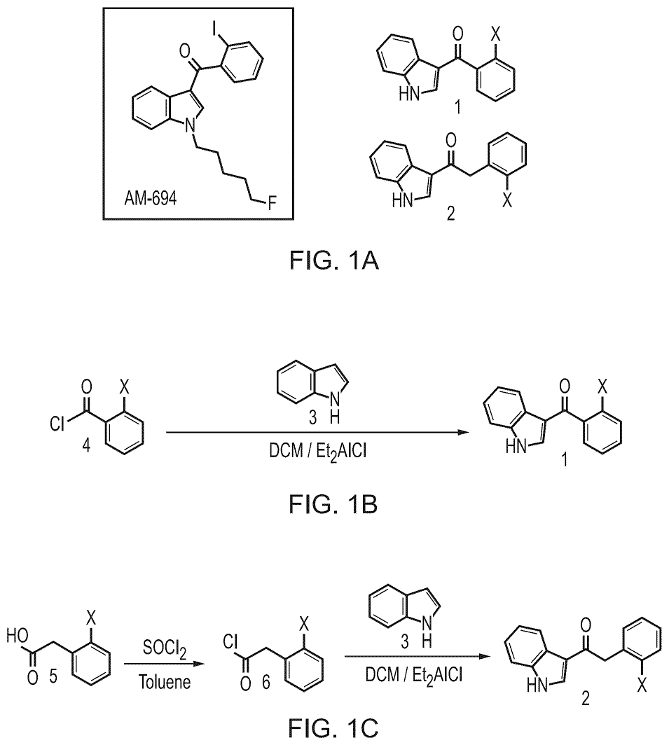

FIG. 1A illustrates schematically a model series of 8 compounds, based on a core, linker, and ring section of AM-694. Compounds 1a-d contain a methanone linker and 2a-d contain an ethanone linker group, all with varied halogen-substitution at the 2-position;

FIG. 1B illustrates schematically synthesis of compounds 1a-d. Atom X denotes halogen substitution (X═F, Cl, Br, I);

FIG. 1C illustrates schematically synthesis of compounds 2a-d. Atom X denotes halogen substitution (X═F, Cl, Br, I);

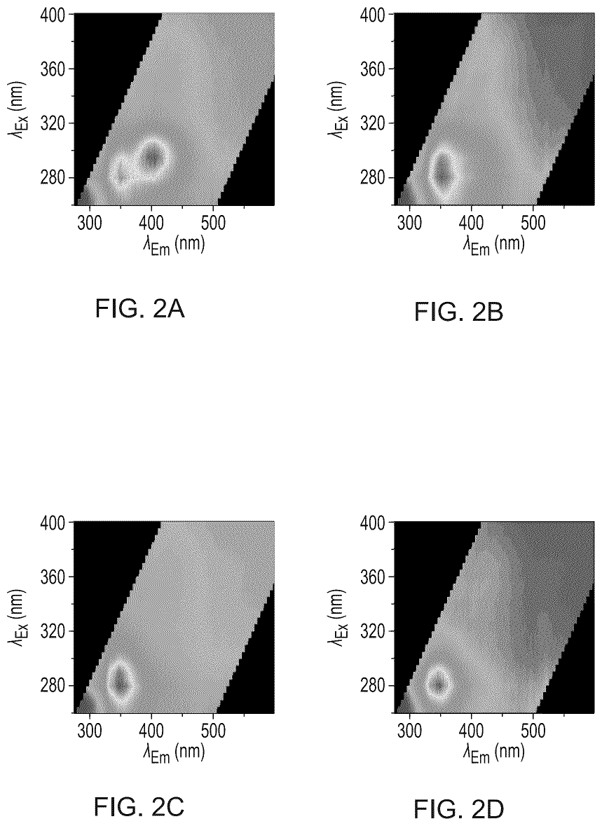

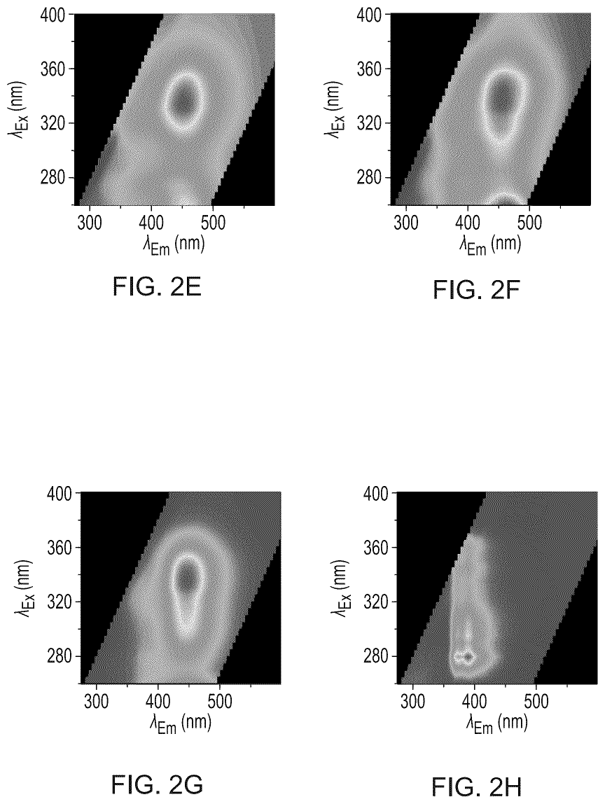

FIG. 2A-D illustrate Fluorescence Spectral Fingerprints (FSFs) for methanone-linked series of SCRA analogues 1a-d (FIG. 2A=1a; FIG. 2B=1b; FIG. 2C=1c; FIG. 2D=1d), collected prior to sample irradiation;

FIG. 2E-H illustrates FSFs for ethanone-linked series of SCRA analogues 2a-d (FIG. 2E=2a; FIG. 2F=2b; FIG. 2G=2c; FIG. 2H=2d), collected prior to sample irradiation;

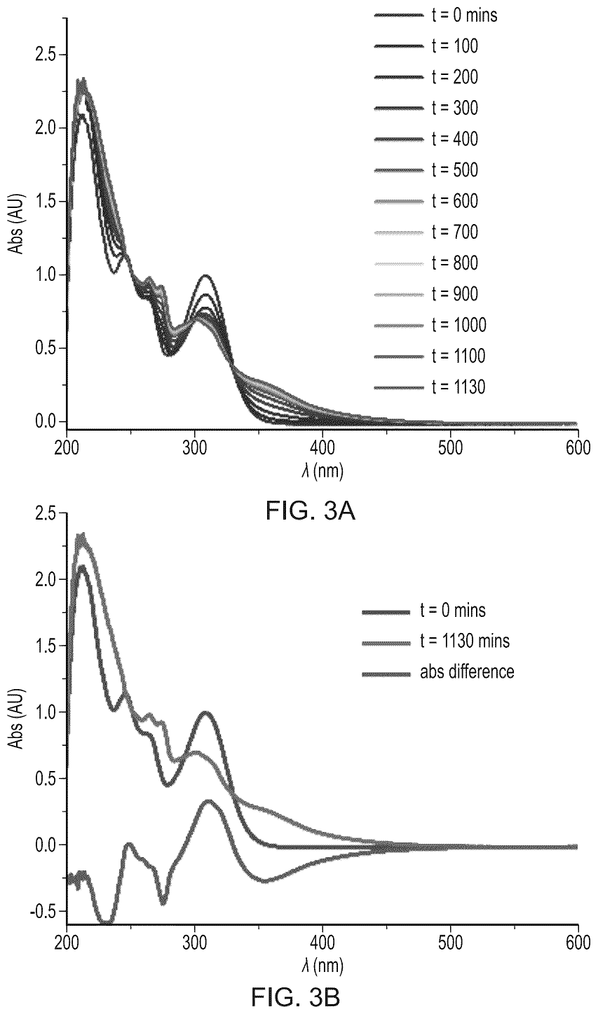

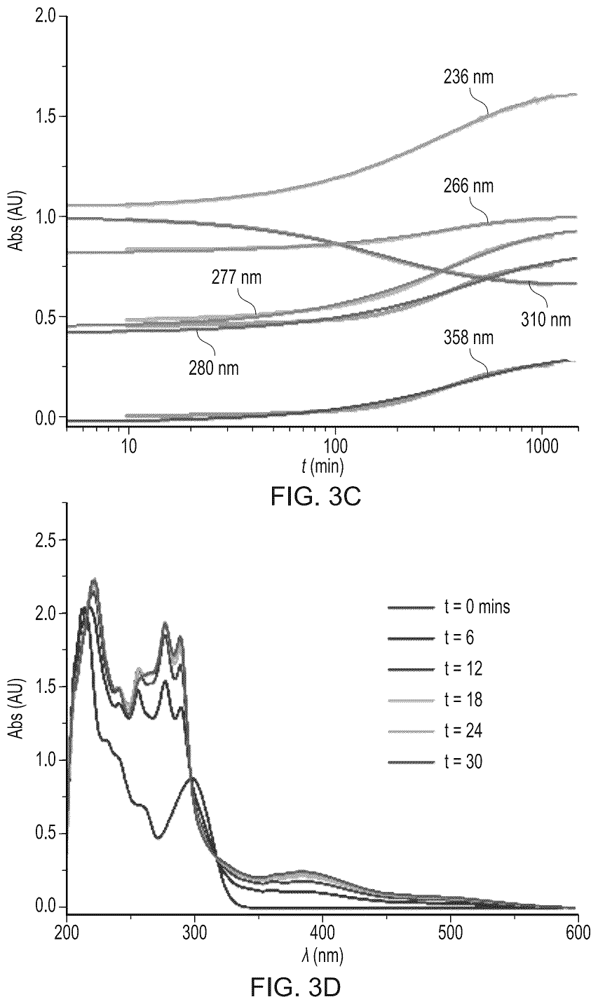

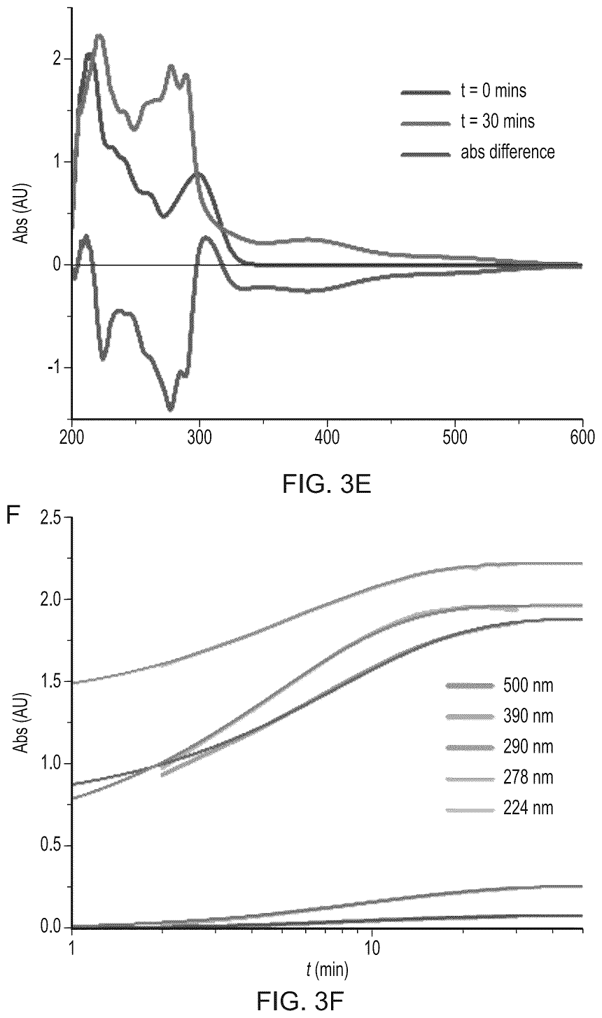

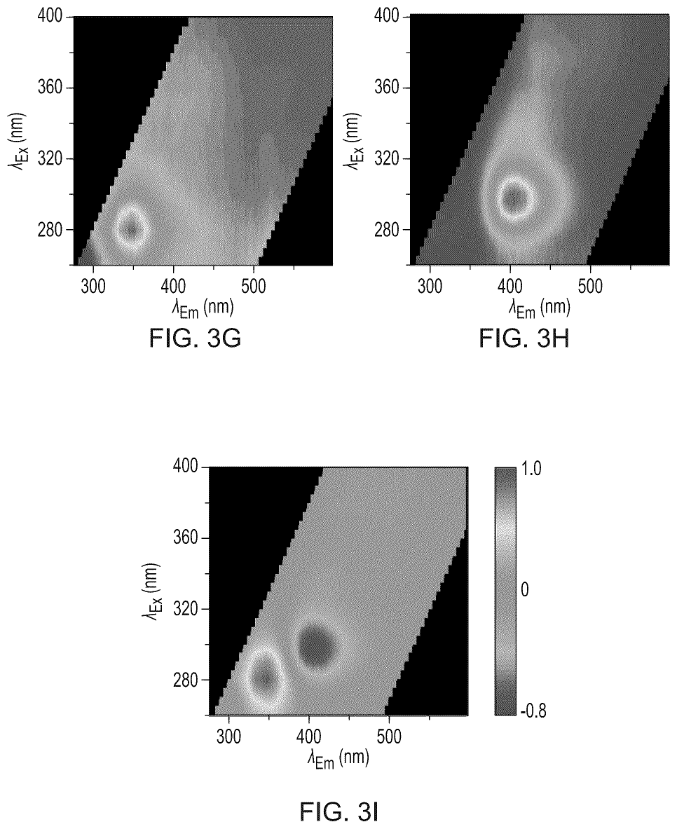

FIG. 3A-C illustrates a time series of absorbance scans of compound 1d as it was degraded (FIG. 3A), a plot comparing the initial and final absorbance spectra (FIG. 3B), the changes in absorbance at wavelengths of interest (FIG. 3C);

FIG. 3D-F illustrates a time series of absorbance scans of compound 2d as it was degraded (FIG. 3D), a plot comparing the initial and final absorbance spectra (FIG. 3E), the changes in absorbance at wavelengths of interest (FIG. 3F);

FIG. 3G-I, show FSFs for 1d collected prior to sample irradiation (FIG. 3G) and post degradation (FIG. 3H), red colouration represents emission with a relative intensity of one, blue an intensity of zero. A heat map (FIG. 3I) showing the differences in emission intensity;

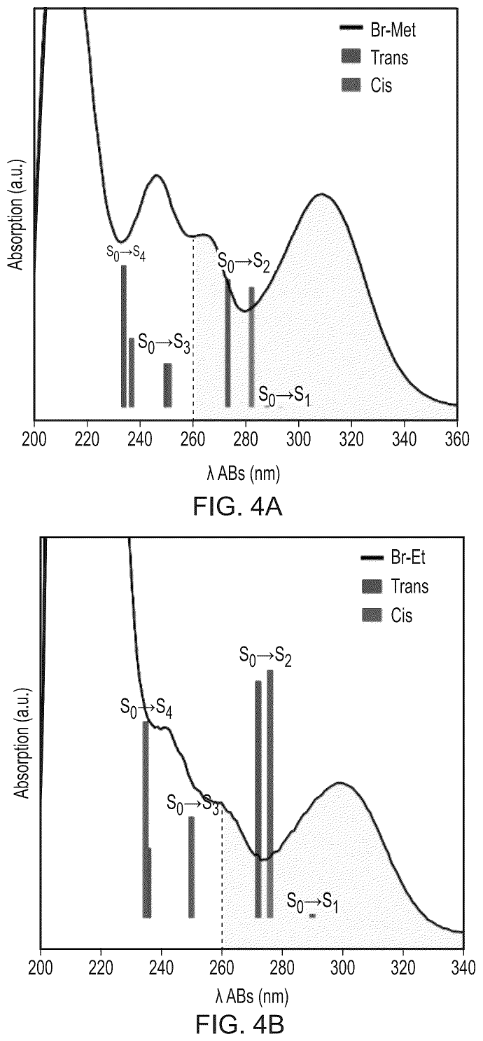

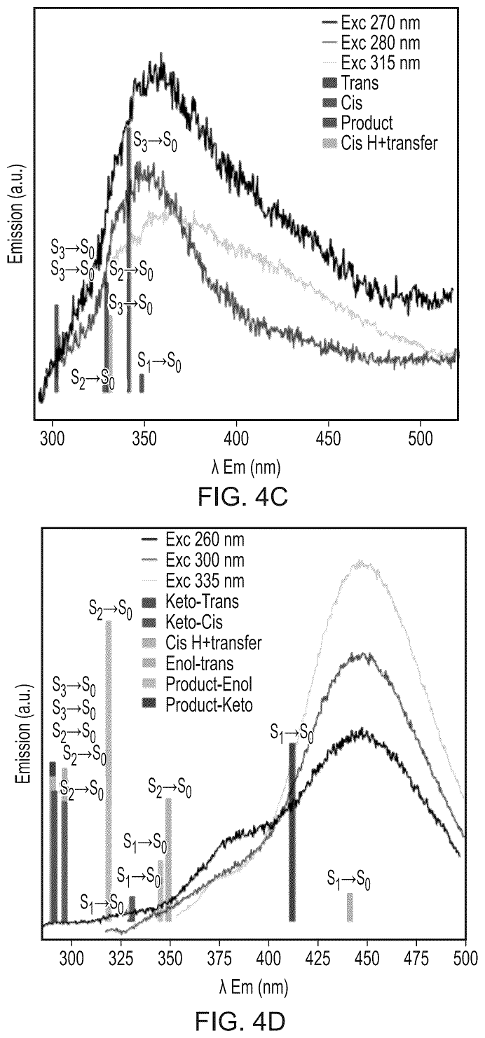

FIG. 4A-B illustrate graphically experimental absorption spectra of compounds 1c and 2c including the assignment of the main bands. The bars represent the computed vertical excitation energies and their computed oscillator strengths. The yellow area represents the experimental excitation wavelengths range used in this work;

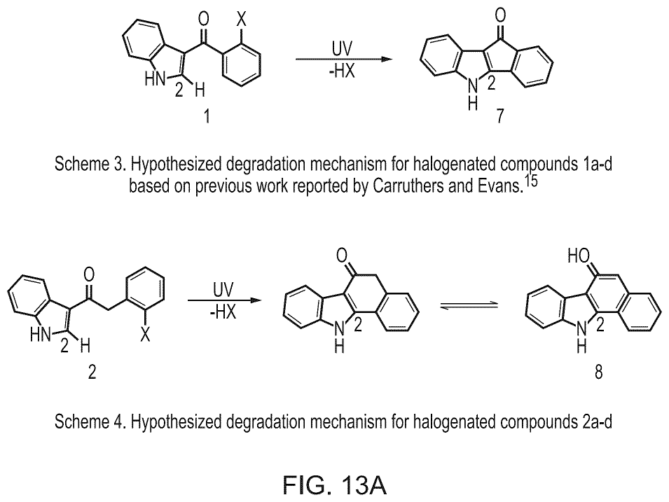

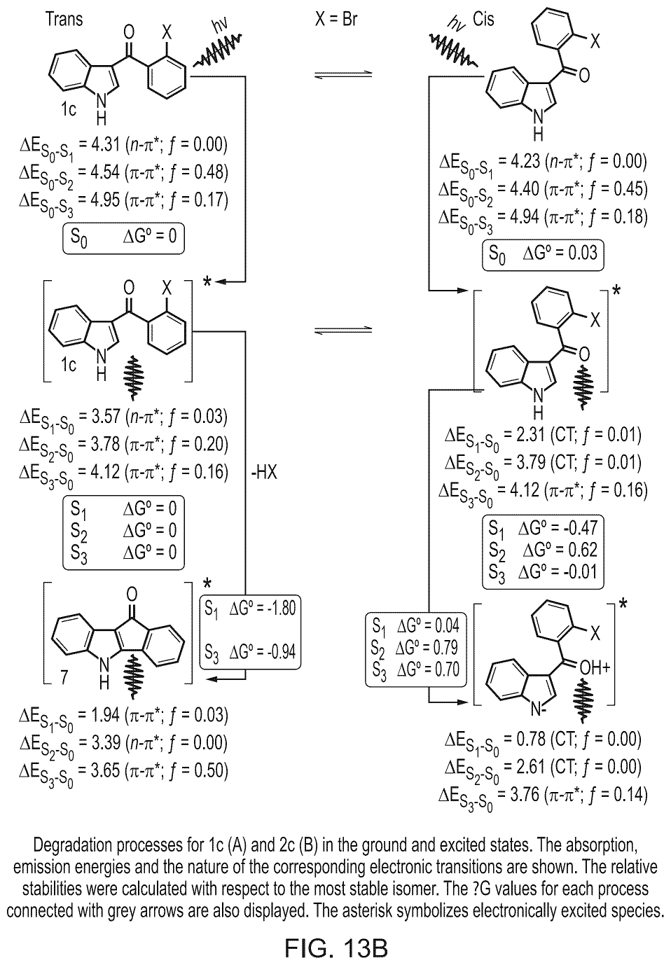

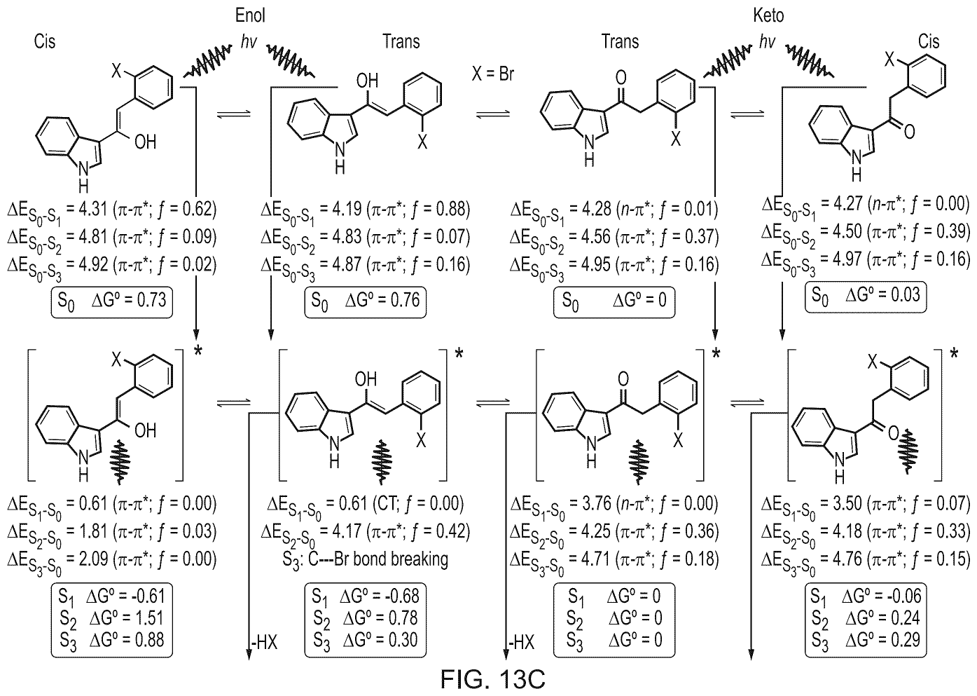

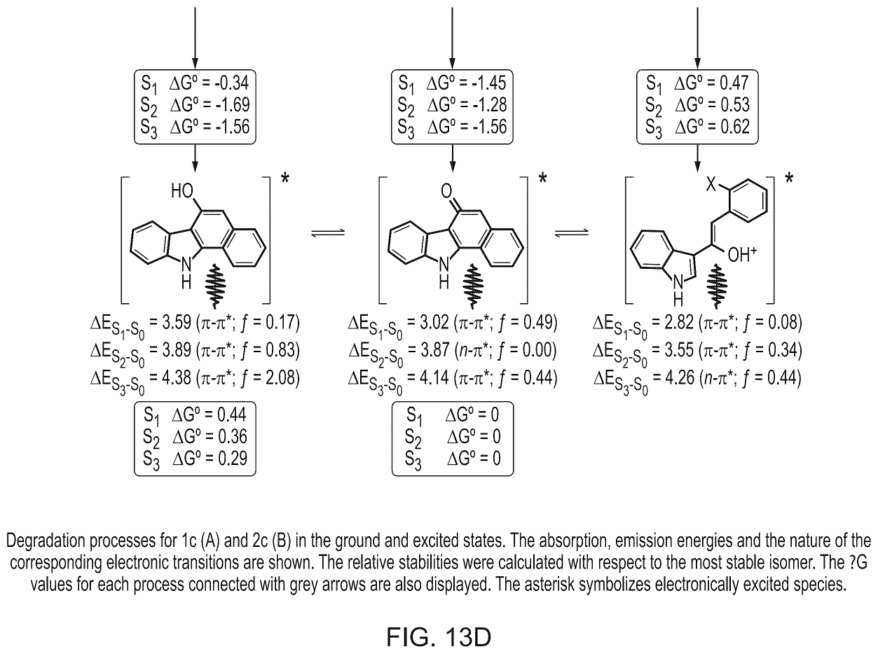

FIG. 4C-D illustrate graphically experimental fluorescence spectra measured in different excitation conditions. The bars represent the vertical emission energies depicted in FIGS. 13B-D and their height are equal to the corresponding oscillator strengths;

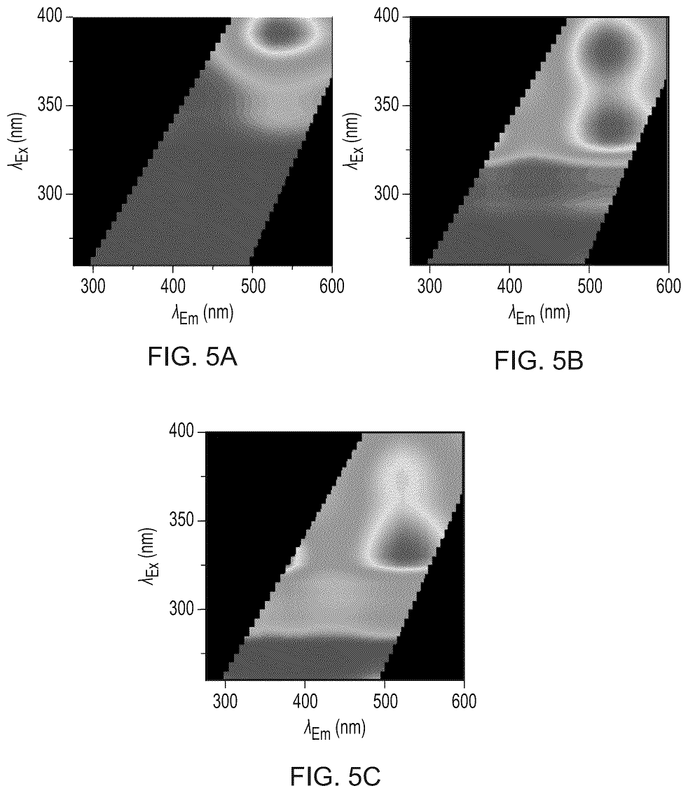

FIG. 5A-C illustrates FSFs for compound 7 (0.2, 0.05 and 0.025 mg/mL, respectively). Red colouration represents emission with a relative intensity of one, and blue represents an intensity of zero. Conditions: Samples in methanol, 20° C., 1 cm path length. Data has not had methanol blank subtracted due to uneven quenching of methanol signal across excitation wavelength and sample concentration ranges;

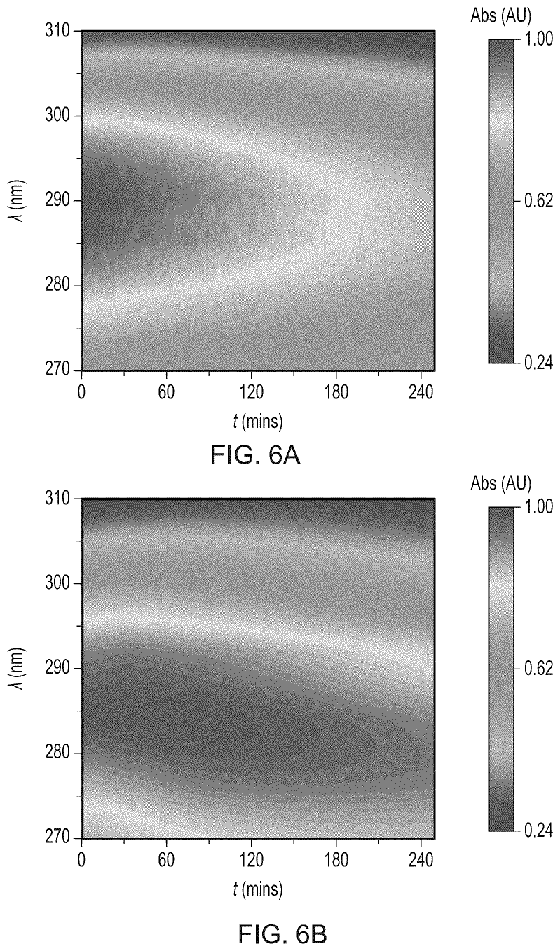

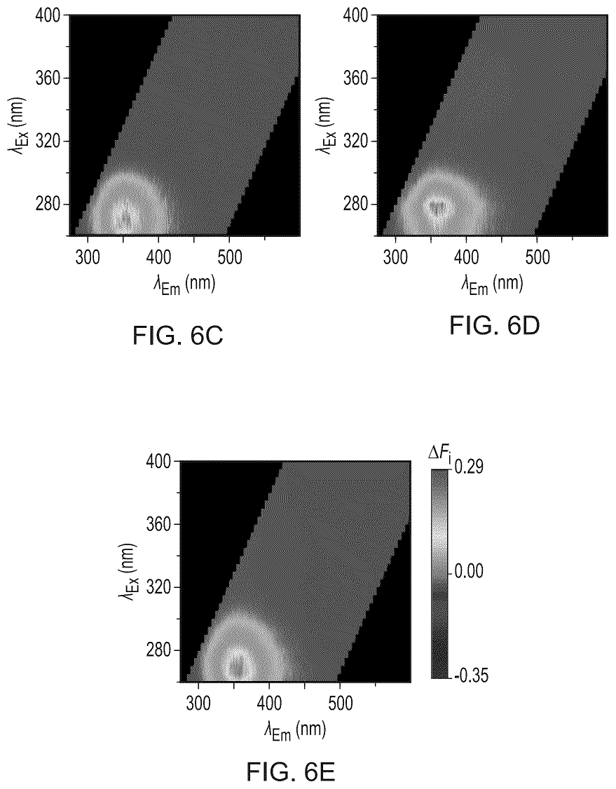

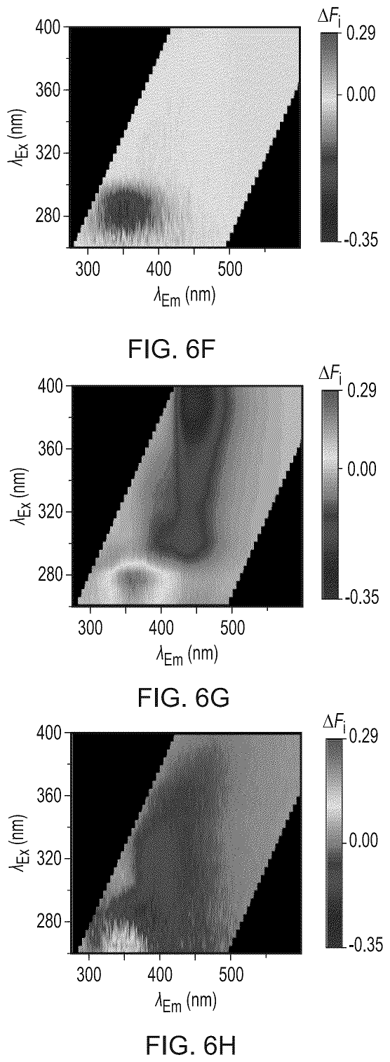

FIG. 6A-B show changes in intensity of the absorbance peak of a control saliva sample (FIG. 6A) and a saliva sample with compound 2d (FIG. 6B) at 1 μg/mL. Red colouration represents emission with a relative intensity of one;

FIG. 6C-E show FSFs for a control saliva sample (FIG. 6C), a saliva sample with compound 1d (FIG. 6D) at 1 μg/mL and a saliva sample with compound 2d (FIG. 6E) at 1 μg/mL, all taken prior to sample irradiation. Red colouration represents emission with a relative intensity of one, blue an intensity of zero;

FIG. 6F-H are differences maps displaying the changes in fluorescent behaviour of samples exposed to intense UV light in: a control saliva sample (FIG. 6F), a saliva sample containing compound 1d (FIG. 6G) and a saliva sample containing compound 2d (FIG. 6H). ΔFi represents the change in signal intensity;

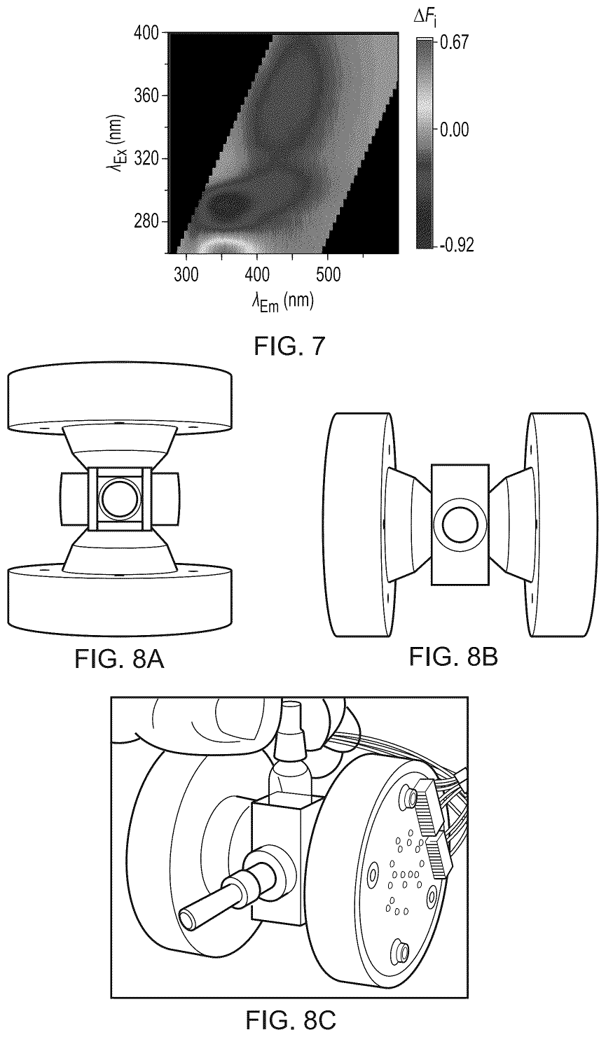

FIG. 7 shows a difference map of FSFs (pre- and post-irradiation) for saliva containing combusted SCRA, AM-694. ΔFi represents the change in signal intensity;

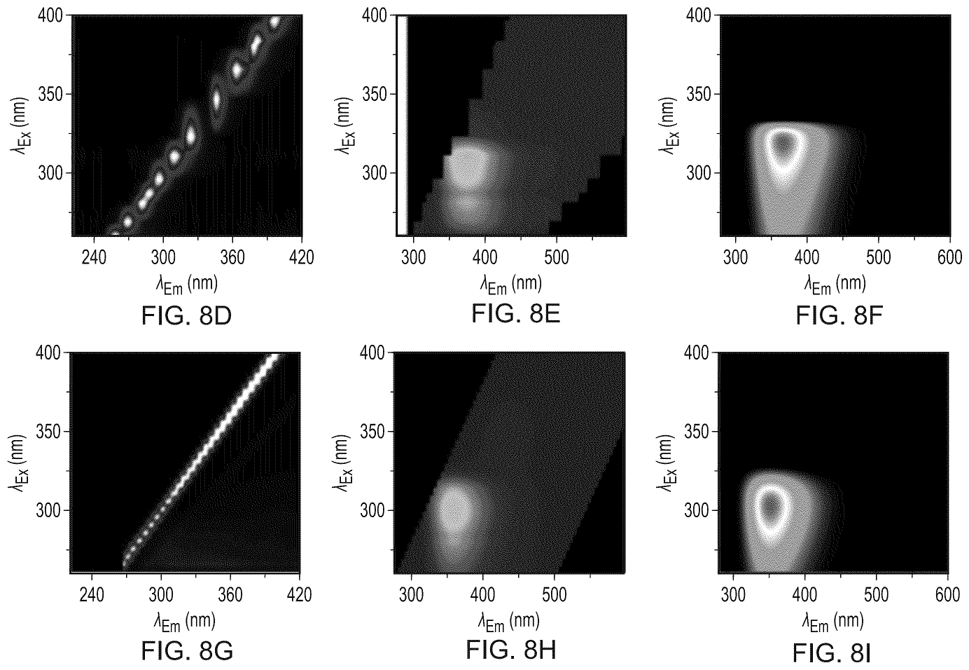

FIG. 8A-B show schematically a prototype portable multi-excitation wavelength fluorimeter. FIG. 8A and FIG. 8B, respectively are top and side views of a 3D printed housing for: a custom cuvette holder; light collecting optics; and LED arrays mounted on custom PCBs. Emitted light from an irradiated sample is collected via a commercially available portable spectrometer;

FIG. 8C illustrates the prototype of FIG. 8A and FIG. 8B in operation.

FIG. 8D and FIG. 8G are respectively a characterisation of LEDs showing the peak positions and spectral bandwidths and the excitation from a monochromator-based, bench-top fluorimeter;

FIG. 8E and FIG. 8H are respectively an EEM of a SCRA (1 μg ml-1 5F-MDMB-PINACA) using the LED-based device and using the bench-top fluorimeter;

FIG. 8F and FIG. 8I are the resultant numerical models (Eq 1) arising from FIG. 8E and

FIG. 8H, respectively. These data shown very close agreement between the parameters extracted from Eq 1;

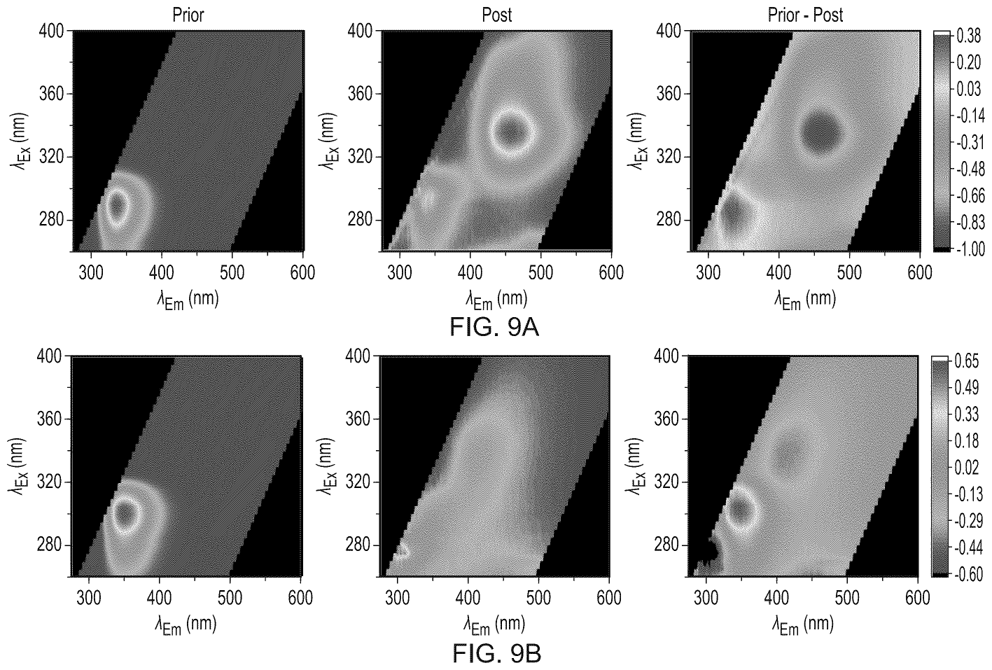

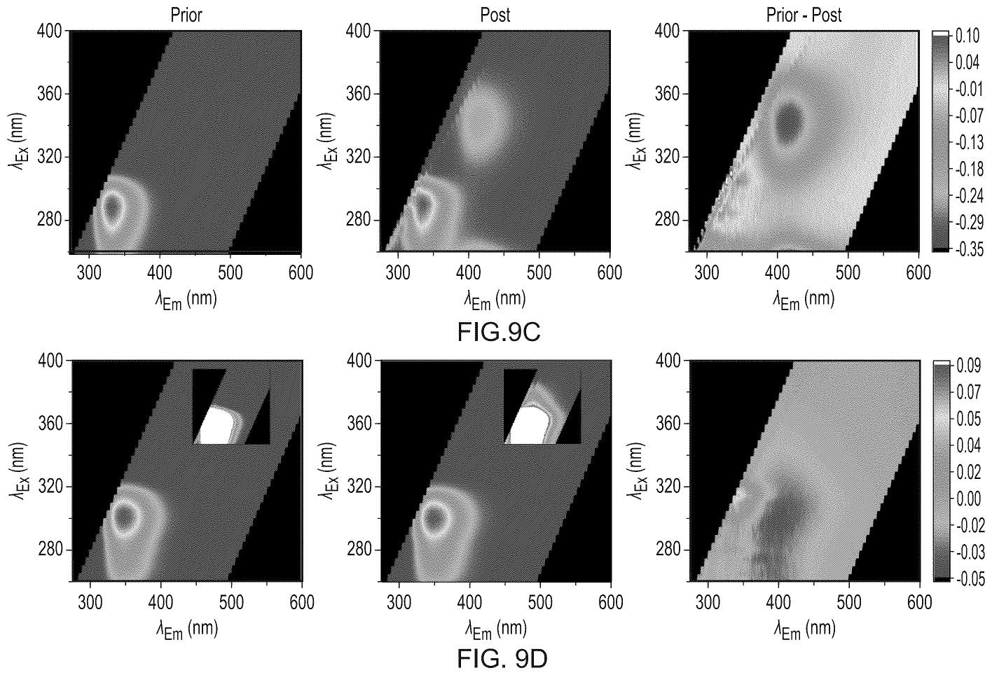

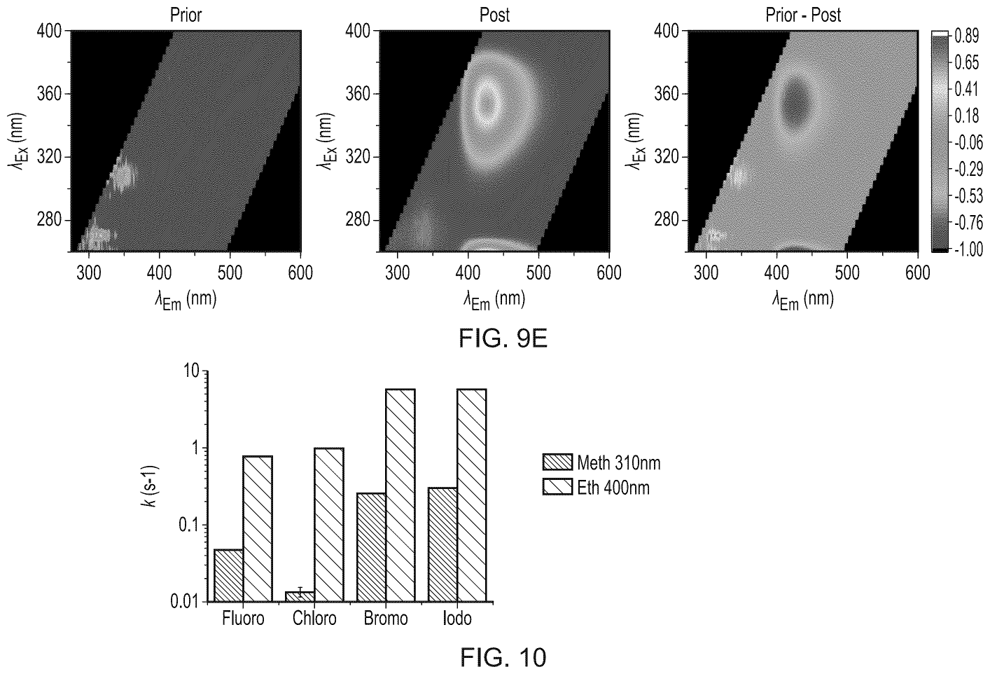

FIG. 9A-E illustrates FSFs for (FIG. 9A) MDMB-4en-PICA, (FIG. 9B) MDMB-4en-PINACA, (FIG. 9C) MDMB-FUBICA, (FIG. 9D) MDMB-FUBINACA and (FIG. 9E) MDA-19 shown prior and post degradation;

FIG. 10 illustrates graphically rate constants for the degradation of the maximum absorbance peak for 1a-d (Meth 310 nm) and 2-d (Eth 400 nm). Note that different spectral bands are selected to prevent spectral convolution in the kinetic data as described herein;

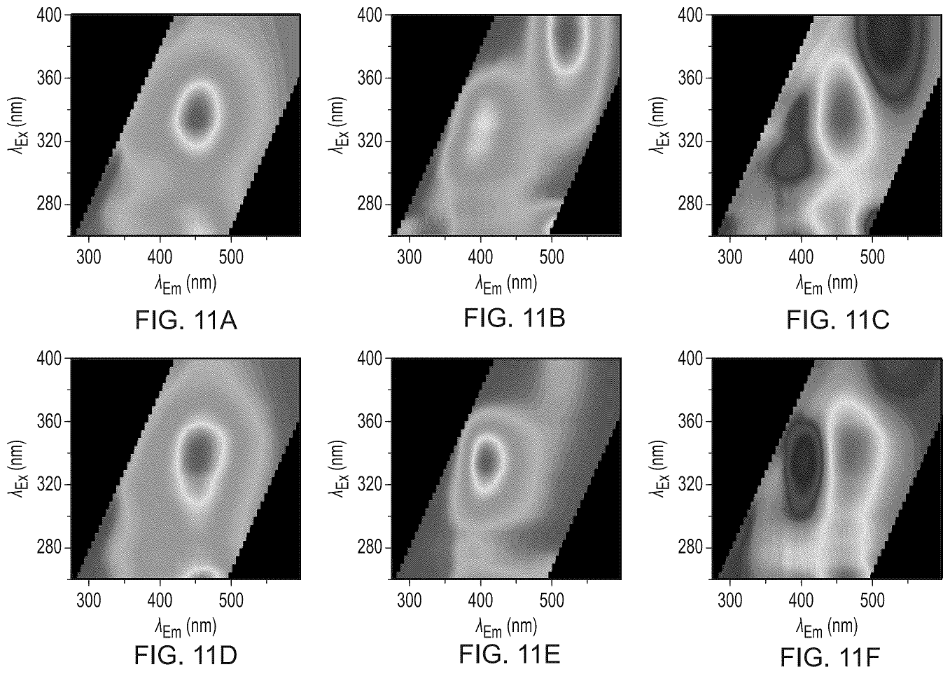

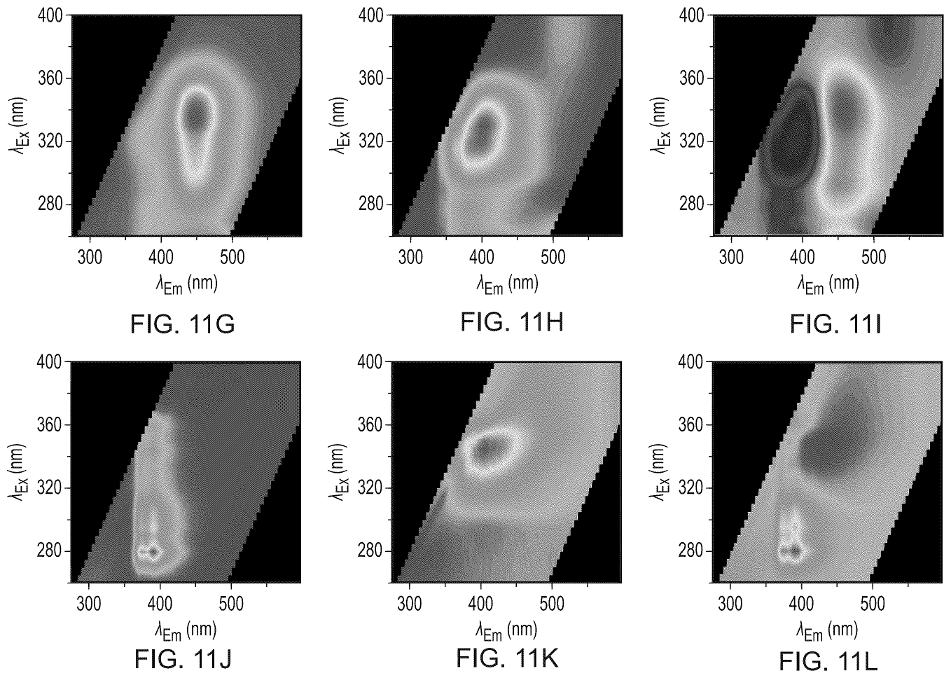

FIG. 11A-L shows FSFs for compound 2a (FIG. 11A-C), compound 2b for (FIG. 11D-F), compound 2c for (FIG. 11G-I) and compound 2d (FIG. 11J-L) those FSFs shown were collected pre and post sample photodegradation. Red coloration represents emission with a relative intensity of one, blue an intensity of zero, and a heat map showing the differences in emission intensity;

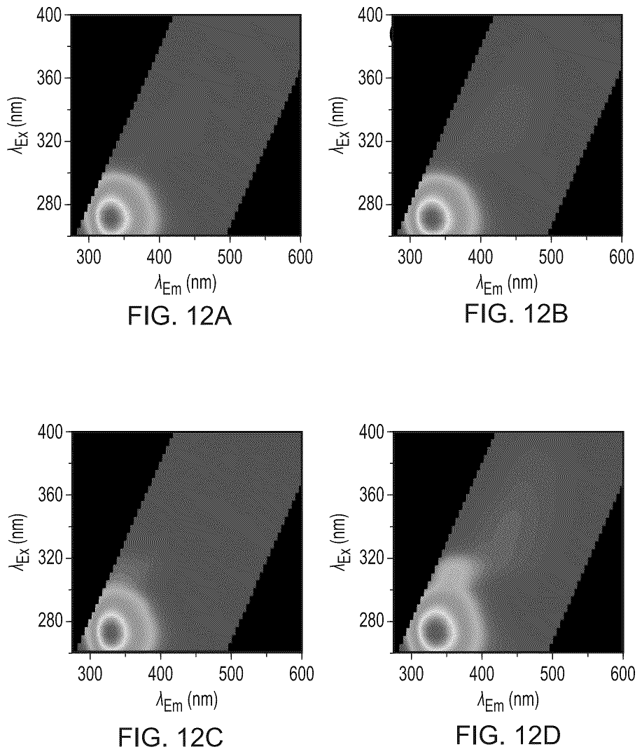

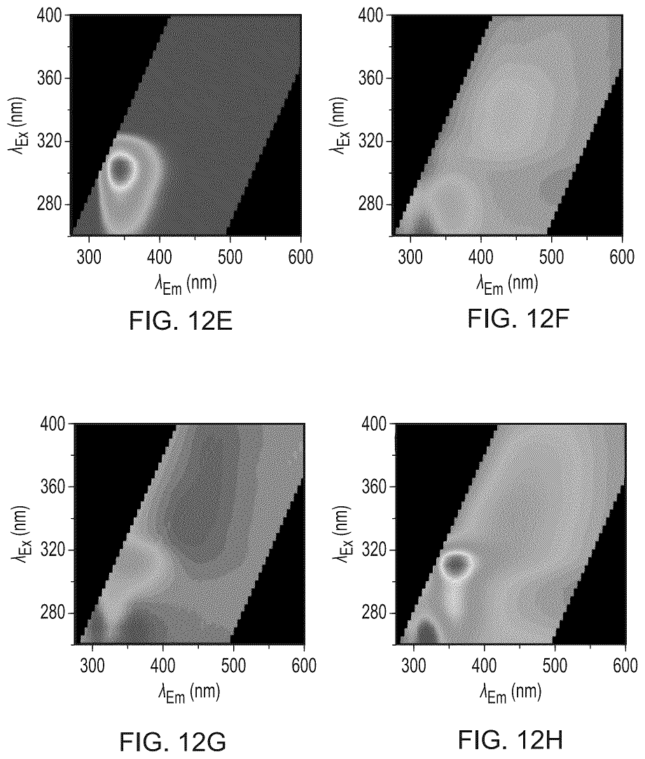

FIG. 12A-H show the FSFs for (FIG. 12A) average of saliva/methanol samples used, (FIG. 12B-D) 10, 50 and 250 ng/mL MDMB-4en-PINACA in saliva/methanol and (FIG. 12E) 250 ng/mL MDMB-4en-PINACA in methanol only. Conditions: 1 mL cuvette, 20° C., 1:2 v/v saliva: methanol. Red colouration represents emission with a relative intensity of one, and blue represents an intensity of zero. (FIG. 12F-H) Difference heat maps of 10, 50 and 250 ng/mL MDMB-4en-PINACA in saliva minus averaged saliva, coloured from −0.05 to 0.228 relative intensity (blue to red);

FIG. 13A-D illustrates schematically: Scheme 3 in FIG. 13A: Hypothesized degradation mechanism for halogenated compounds 1a-d based on previous work reported by Carruthers and Evans [15], Scheme 4 in FIG. 13A: Hypothesized degradation mechanism for halogenated compounds 2a-d, and in FIG. 13B-C. Degradation processes for 1c (A, FIG. 13B) and 2c (B, FIG. 13C-D) in the ground and excited states. The absorption, emission energies and the nature of the corresponding electronic transitions are shown. The relative stabilities were calculated with respect to the most stable isomer. The AG values for each process connected with grey arrows are also displayed. The asterisk symbolizes electronically excited species;

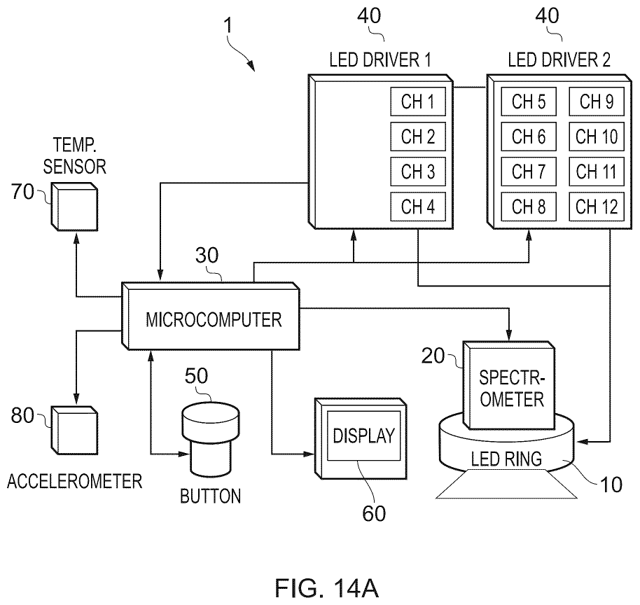

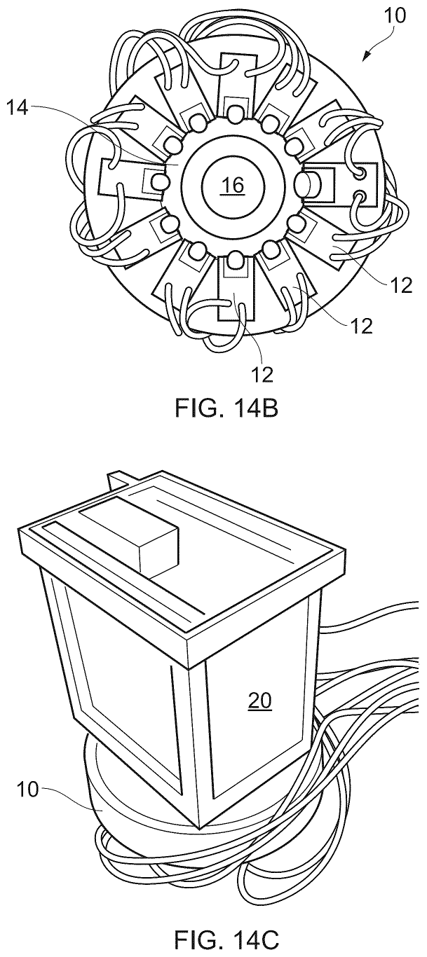

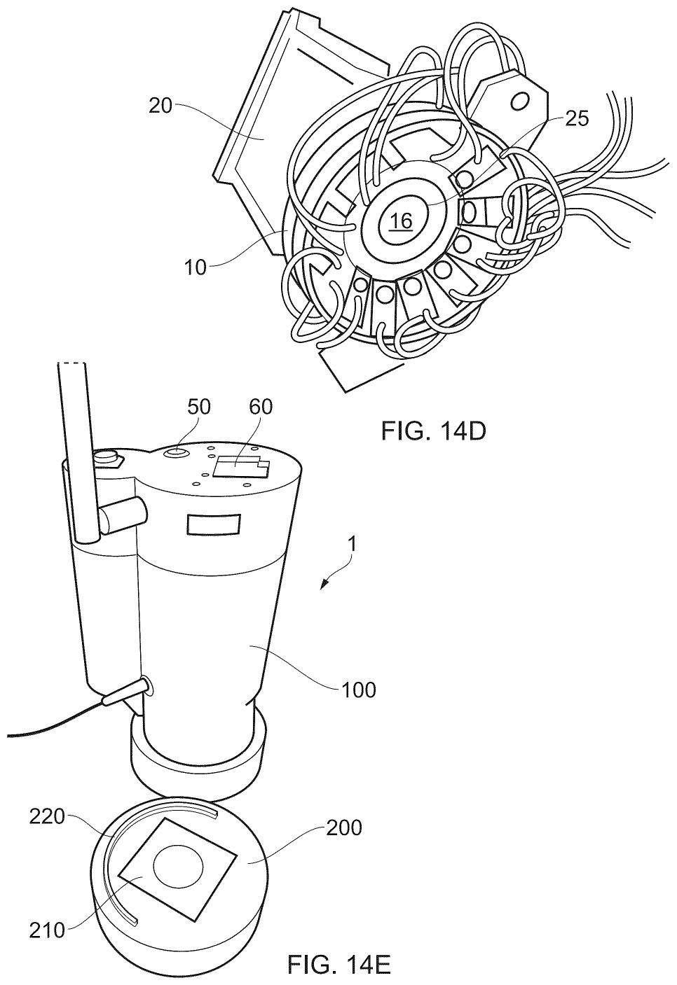

FIG. 14A-E illustrate various aspects of an apparatus configured to perform a hybrid photochemical fingerprinting method;

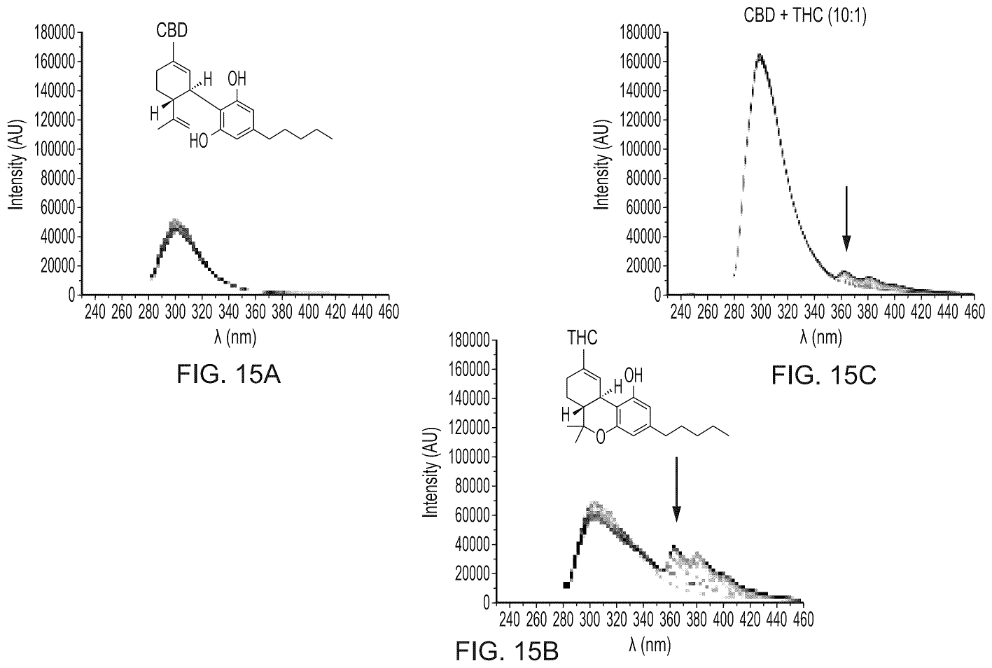

FIG. 15A-C show graphically the emission spectra of CBD, THC and a combination irradiated over time with a 265 nm irradiation source to induce a photochemical change;

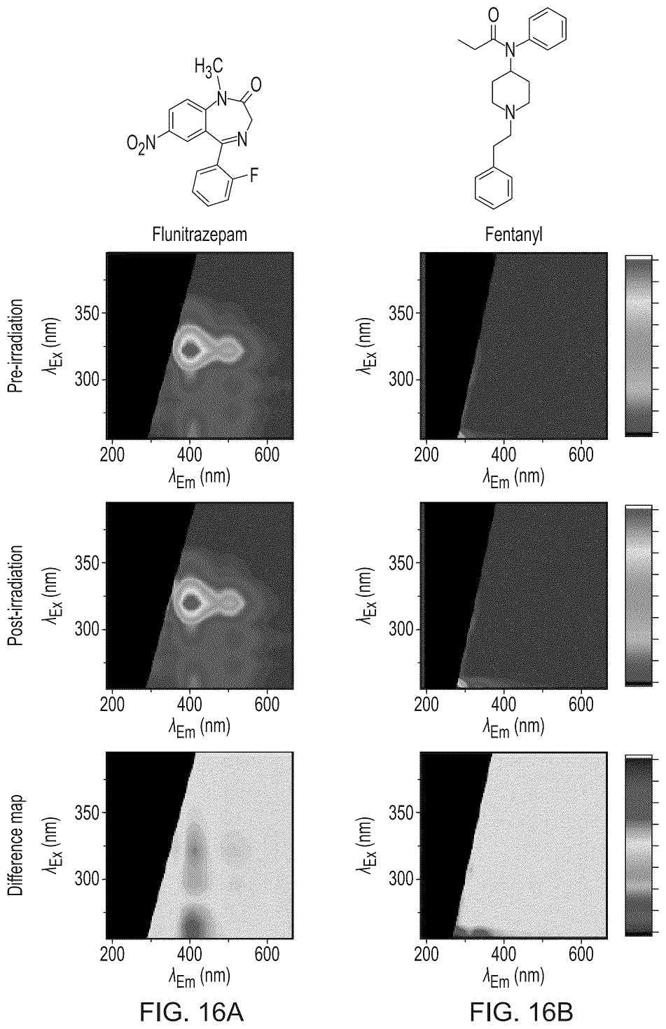

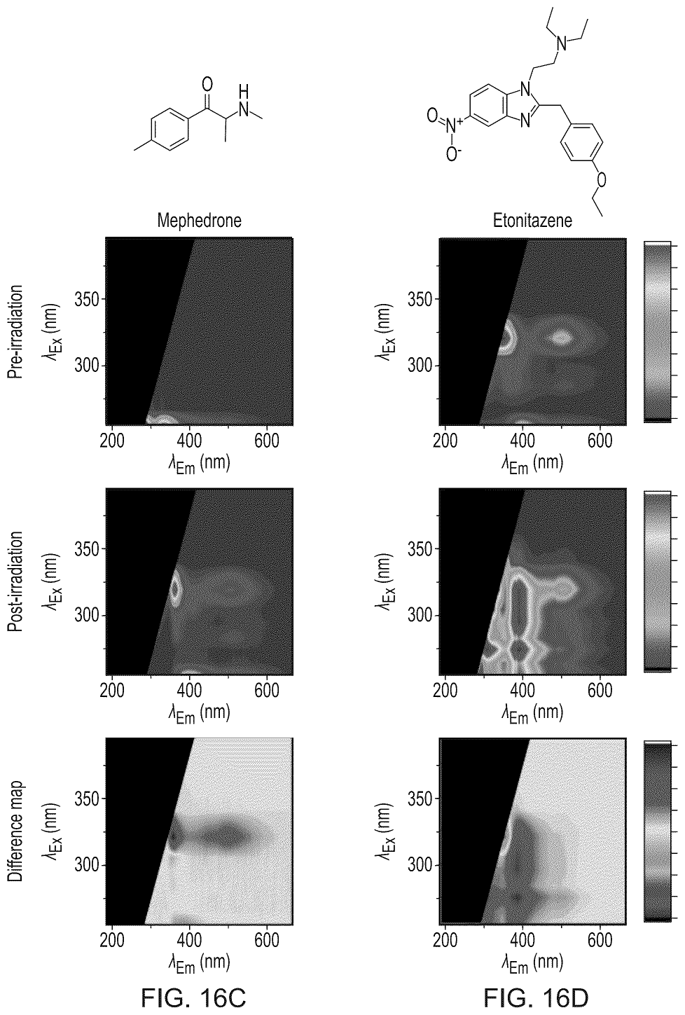

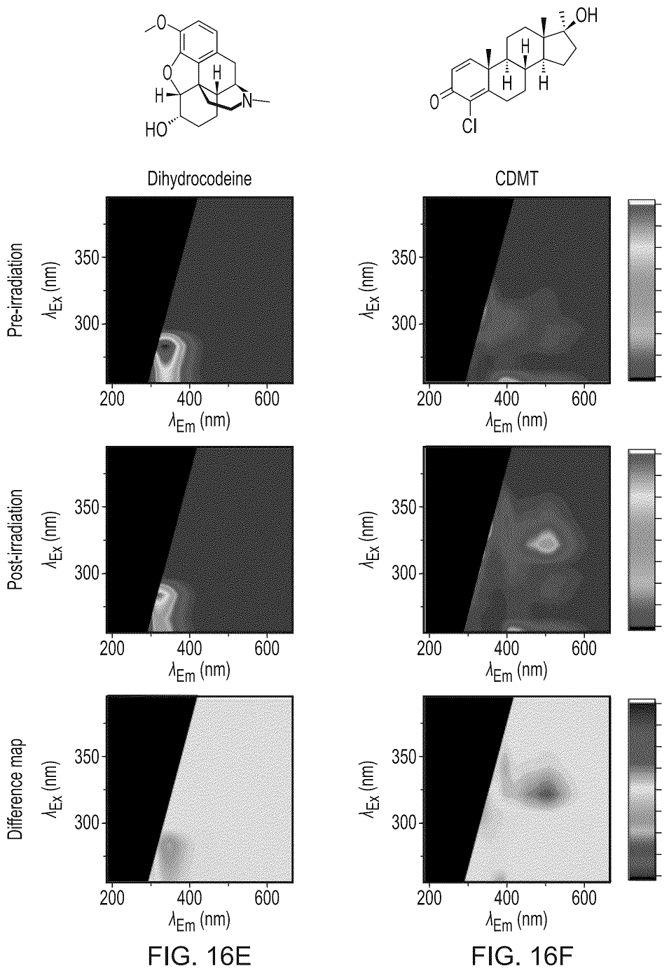

FIG. 16A-F shows fluorescence spectral fingerprints (FSFs) obtained pre- and post-irradiation to induce a photochemical change, together with a difference map comparison between the FSFs for 6 different molecules (Flunitrazepam, Fentanyl, Mephedrone, Etonitizene, Dihydrocodiene and CDMT in each column left to right) tested in an ethanol solution;

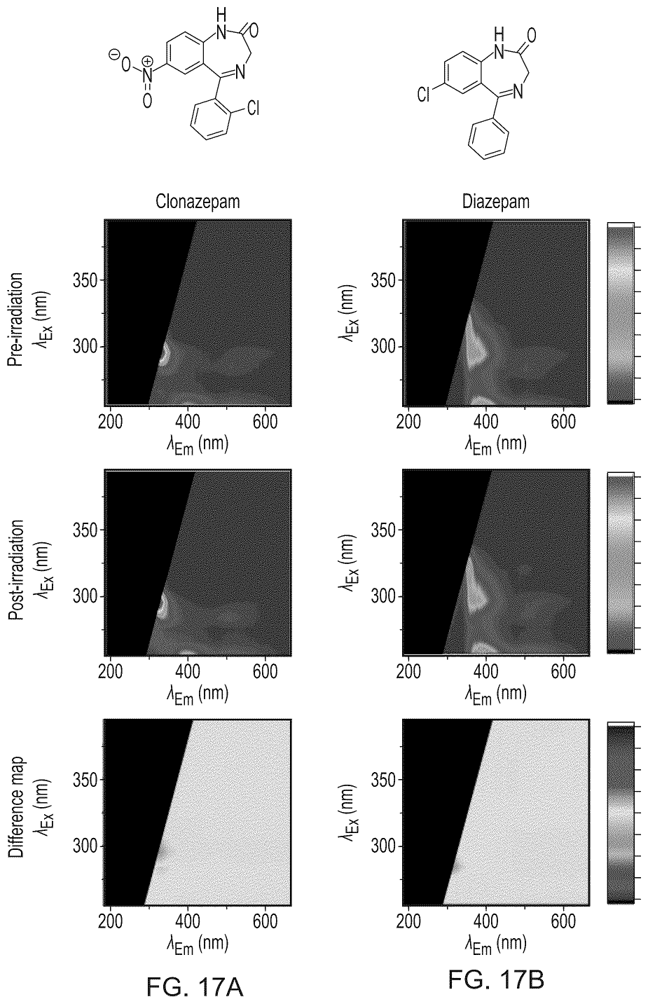

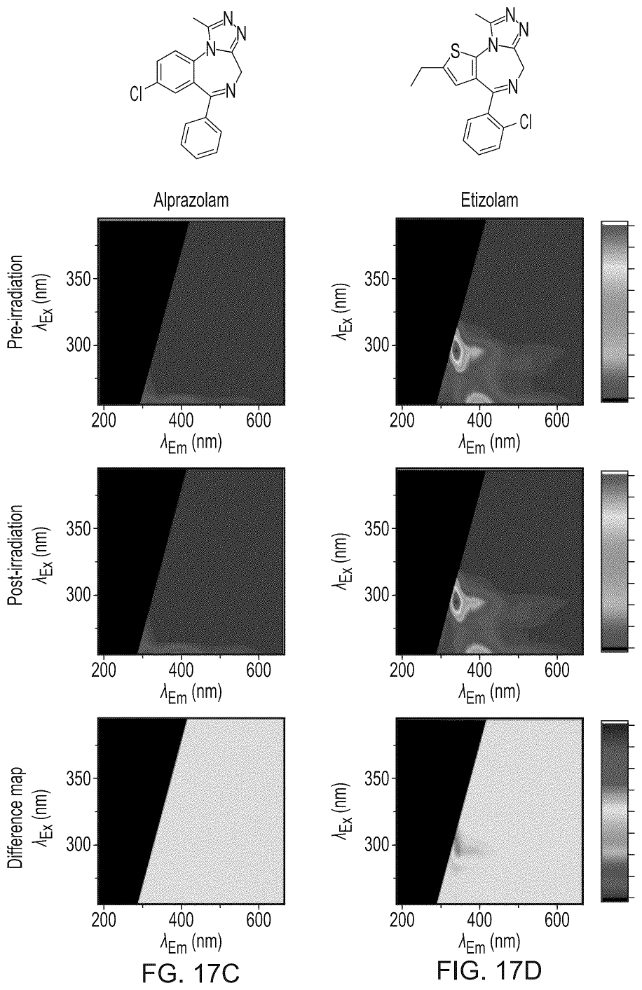

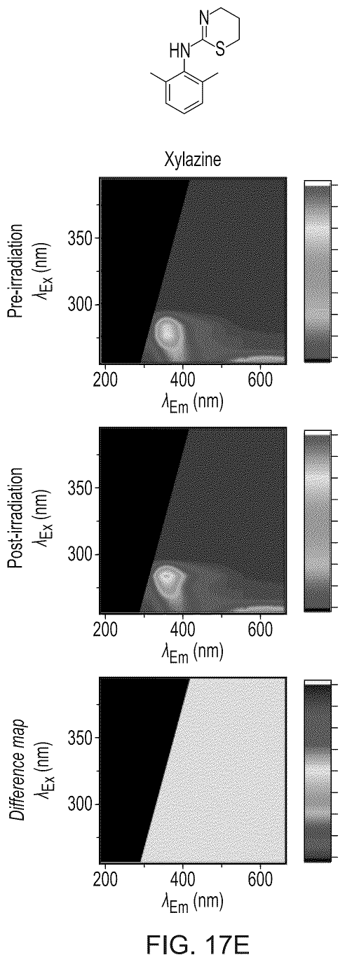

FIG. 17A-E shows fluorescence spectral fingerprints (FSFs) obtained pre- and post-irradiation to induce a photochemical change, together with a difference map comparison between the FSFs for 5 different molecules (Clonazepam, Diazepam, Alprazolam, Etizolam, and Xylazine in each column left to right) tested in an ethanol solution;

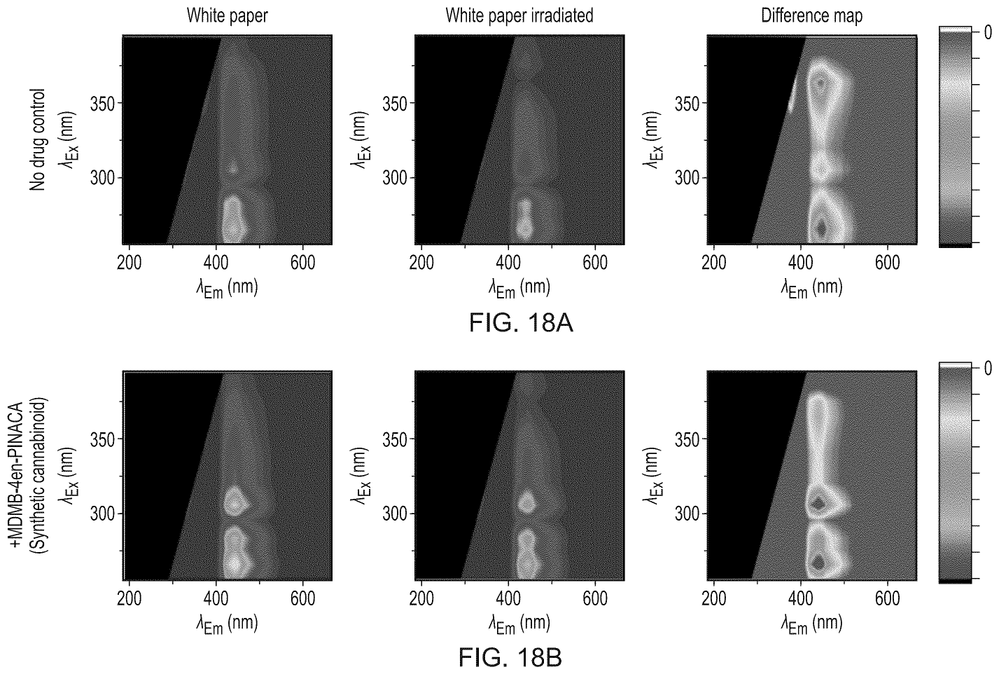

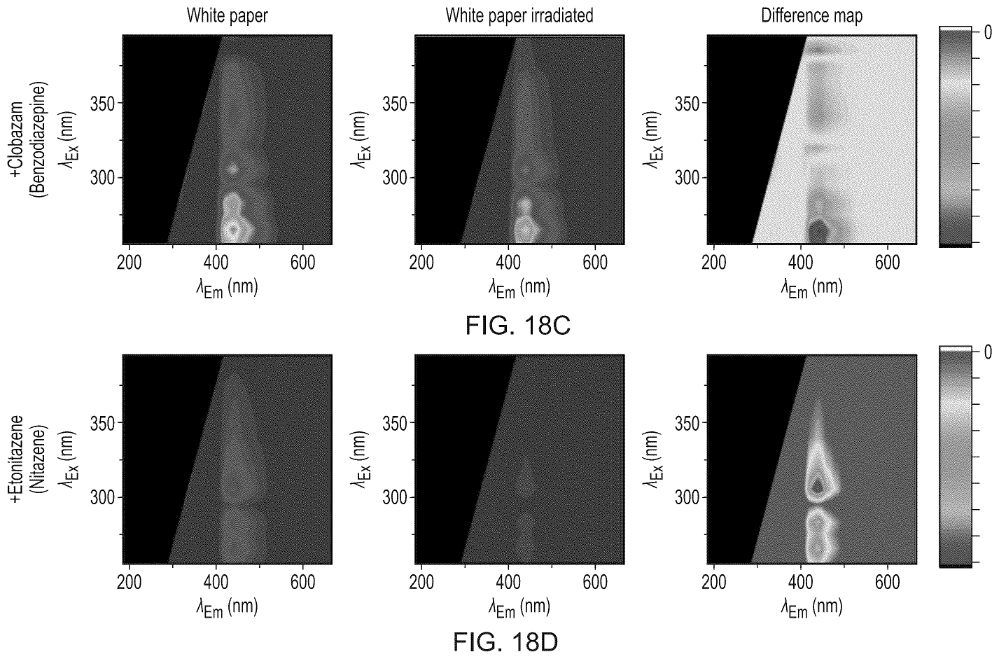

FIG. 18A-D shows fluorescence spectral fingerprints (FSFs) obtained pre- and post-irradiation to induce a photochemical change, together with a difference map comparison between the FSFs for 4 different cases: no drug adsorbed onto a paper matrix; a synthetic cannabinoid adsorbed onto a paper matrix; a Benzodiazepine adsorbed onto a paper matrix; and a Nitazene adsorbed into a paper matrix. Each row relates to one case;

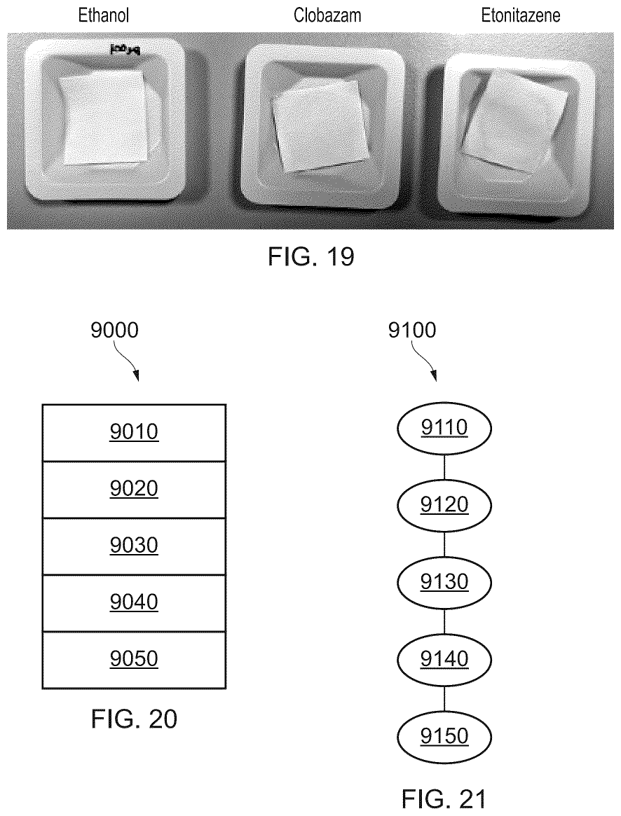

FIG. 19 shows a photograph of samples on paper after irradiation to induce a photochemical change;

[symbol]

FIG. 20 illustrates schematically some components of an apparatus according to some arrangements; and

FIG. 21 illustrates schematically steps of methods performed in accordance with some arrangements.

### DETAILED DESCRIPTION

Before discussing the example embodiments in any more detail, first an overview will be provided.

### Synthetic Cannabinoid Receptor Agonists (SCRAs)

Synthetic cannabinoid receptor agonists (SCRAs), colloquially known as ‘spice’, are a class of designer recreational drugs, commonly taken to mimic the effects of tetrahydrocannabinol (THC). Initially, such drugs were synthesized to be cannabimimetic and potentially provide pain relief to some users. However, additional psychoactive side effects have rendered SCRAs generally unsuitable for pharmaceutical use [1].

A first new SC compound was identified in seized drug samples in 2008. By the end of 2021, there were 224 SCRA compounds formally notified by the European Monitoring Centre for Drugs and Drug Detection (EMCDDA) [2]. In order to circumvent legislation which bans novel psychoactive substances (NPS), more structurally diverse compounds are released into circulation every year [3].

Newer SCRA varieties exhibit higher affinity for CB1 and CB2 receptors, meaning that the newer varieties pose an increasing threat to users with fatal side effects including, but not limited to: coronary artery thrombosis, ischemic stroke and psychosis [4].

### Drug Detection

Detection of SCRA compounds is challenging, with routine testing methods unable to identify the presence of many SCRAs [3].

Point-of-care drug testing is an important modality to support users. Advantageously, detection methods for point-of-care drug testing are both fast and portable. A variety of drug testing methods are available including: screening, colorimetric detection, immunochemical assays, and chromatographic methods. Whilst there are advantages and disadvantages to each method, colorimetric detection is typically favoured in a point of care setting due to being both rapid and portable. However, colorimetric detection tends to be specific to individual structures, and can fail to detect newer SCRAs such as, for example, Cumyl-PEGACLONE [5]. In contrast, chromatographic methods including Liquid Chromatography Mass Spectroscopy (LC-MS) and Mass Spectroscopy (MS) provide a more advanced detection method capable of resolving a large range of compounds, with a low limit of detection [6]. However, the high associated costs and lack of portability renders chromatographic methods generally unsuitable for mobile drug testing.

### Fluorescence Spectral Fingerprinting (FSF)

It has been demonstrated that fluorescence spectral fingerprinting (FSF) has potential as a rapid point-of-care test for SCRAs [7]. FSF may be used, for example, as a probe of SCRA use. It has been shown that common SCRA compounds, in both pure samples and in oral fluid (saliva), produce individual FSFs, with a possibility to extract information about the structure and concentration of these substances from the FSF [7]. It has been suggested that part of the sensitivity of SCRA FSFs to different, structurally similar, molecules may arise from differences in cross conjugation and associated effects on electronic transitions related to fluorescence [7].

### Enhanced Fluorescence Spectral Fingerprinting (FSF)

Described arrangements recognise that the rapid detection of SCRAs can be enhanced by augmenting a FSF detection methodology through photochemical reactivity tracking.

Such an approach recognises that SCRAs are typically built on a scaffold that includes a central ‘core’ group. There are over 10 different moieties that have been identified as core groups in SCRA compounds including: pyrrole, carbazole and more recently, oxoindole found in the emerging “OXIZID” SCRA group [8], [9]. However, indole and indazole are by far the most commonly identified core in SCRA compounds, found in over 75% of SCRAs notified by the EMCDDA [8]. Both indole and indazole are photochemically active and are sensitive to substituents on the ring system [10].

As described below, a model SCRA homologue series is used to explore the molecular determinants of SCRA FSF sensitivity and the potential for tracking photochemical reactivity of SCRAs via changes in FSF for enhanced detection. Density-functional theory (DFT) and time-dependent DFT (TDDFT) calculations suggest a molecular rationale for the detection sensitivity. The feasibility of using a combined photochemical/FSF ‘photochemical fingerprinting’ approach to detect street material in saliva is demonstrated.

### Approaches and Methods

### (i) Exploring the Sensitivity of SCRA FSFs.

Previous work suggests that SCRA FSFs are highly sensitive to chemical substitution, attributed to changes in electronic structure and the degree of cross-conjugation. Therefore, a model chemical series has been designed to explore these perturbations.

FIG. 1A-C illustrates schematically a model series of 8 compounds, based on a core, linker, and ring section of AM-694. Compounds 1a-d contain a methanone linker and 2a-d contain an ethanone linker group, all with varied halogen-substitution at the 2-position. These compounds are based on the SCRA, AM-694, although excluding the fluoropentyl ‘tail’ on the indole nitrogen for synthetic simplicity. Moreover, ‘tail-less’ SCRAs have recently been reported (e.g. MDMB-5Br-INACA) and so the model system is both experimentally tractable and relevant as an example SCRA.

All eight of the model series set out in FIG. 1A-C were successfully synthesized via a selective acylation of indole (3) at the 3-position, in the presence of the Lewis acid, diethylaluminium chloride (FIG. 1A-C). All eight compounds vary by their halogen substitution at the 2-position on the benzene ring, and whether the indole is attached to the halogenated phenyl with a methanone or ethanone linker group. All eight compounds were characterized using ¹H, ¹³C and ¹⁹F (where applicable) NMR, mass spectrometry, IR spectroscopy and melting point (data in Supporting Information, SI below).

FIG. 2A-H shows the resulting FSFs for all eight compounds, 1a-d and 2a-d. These data show each FSF varies between linker groups and halogen substitution, and that these shifts are complex. In particular, FIG. 2A-D illustrate Fluorescence Spectral Fingerprints (FSFs) for methanone-linked series of SCRA analogues 1a-d (FIG. 2A=1a; FIG. 2B=1b; FIG. 2C=1c; FIG. 2D=1d), collected prior to sample irradiation; and FIG. 2E-H illustrate FSFs for ethanone-linked series of SCRA analogues 2a-d (FIG. 2E=2a; FIG. 2F=2b; FIG. 2G=2c; FIG. 2H=2d), collected prior to sample irradiation. In FIG. 2A-H, red colouration represents emission with a relative intensity of one, and blue represents an intensity of zero. Conditions: 0.01 mg/mL in methanol, 20° C., 1 cm path length.

The broad trends shown in FIG. 2A-H are discussed below:

It has previously been found that fitting SCRA FSFs with a modified Fraser-Suzuki function allows an accurate quantification of relatively complex spectral fingerprints [7].

$$Fi = \qquad [ Equation 1 ]$$

$${F}_{0} + \sum A exp [ - \frac{ln 2}{{b}_{1}^{2}} {( ln \frac{1 + 2 {b}_{1 ( {λ}_{Em} - {λ}_{Em}^{max} )}}{{w}_{1}} )}^{2} - \frac{ln 2}{{b}_{2}^{2}} {( ln \frac{1 + 2 {b}_{2 ( {λ}_{Ex} - {λ}_{Ex}^{max} )}}{{w}_{2}} )}^{2} ]$$

Equation 1 is a sum of two-dimensionally skewed Gaussian functions, where:
A is the amplitude
w the full width at half-maximal (FWHM) and
b is a skewness parameter.

In practice it has been found that the FSFs for 1a-d and 2a-d are accurately captured with either one or two components and the resulting fit parameters are given in Table S1.

TABLE S1
Mean ± Standard Deviation of the position parameters
taken from the models fit to FSFs of the SCRA analogues (Eq1).

| Molecule | λ_{Em}1 (nm) | λ_{Ex}1 (nm) | λ_{Em}2 (nm) | λ_{Ex}2 (nm) |
|---|---|---|---|---|
| 1a | 403 ± 0.9 | 291 ± 0.3 | 348 ± 0.3 | 281 ± 0.1 |
| 1b | 356 ± 2.5 | 280 ± 0.5 | 417 ± 14 | 360 ± 7 |
| 1c | 347 ± 0.7 | 281 ± 0.3 | 410 ± 2.3 | 343 ± 3.3 |
| 1d | 347 ± 1.8 | 279 ± 0.6 | 411 ± 9 | 263 ± 1.2 |
| 2a | 453 ± 0.6 | 336 ± 0.6 | 453 ± 5.0 | 264 ± 1.0 |
| 2b | 451 ± 1.4 | 323 ± 0.7 | 462 ± 0.5 | 255 ± 3.8 |
| 2c | 448 ± 0.1 | 335 ± 0.7 | — | — |
| 2d | 384 ± 0.3 | 282 ± 1.4 | — | — |

Compounds 1a-d (FIG. 2A-D) each show a major spectral feature at λ_{Ex}^{max}≈280 nm and λ_{Em}^{max}≈350 nm, whereas 2a-d (FIG. 2E-H) each have a major component at λ_{Ex}^{max}≈325 nm, and λ_{Em}^{max}═450 nm, with the notable exception of 2d. That is, the addition of a methylene-bridge carbon at the linker region is sufficient to shift the λ_{Ex}^{max} by ≈80 nm, but without a similar dramatic change in Stokes shift, ≈85 nm. The addition of this extra carbon to the linker region drives the FSFs to be effectively unrecognizable as typical indole fluorescence. Similar ‘complex’ FSFs for SCRAs have previously been observed and it is suggested that these large shifts might arise from changes in cross-conjugation between the two ring systems [7].

Moreover FIG. 2A-H illustrates that there are clear differences associated with varying the halogen substitution, both with methanone and ethanone linked compounds. Broadly, increasing the electronegativity of the halogen substitution from iodine to fluorine causes a blue shift in the λ_{Em}^{max} for all compounds 1a-d and 2a-d, though this is accompanied by a non-obvious trend in λ_{Ex}^{max} (Table S1). Most notably, the observation of measurable shifts in the FSFs on halogen substitution suggests the fluorescence of the indole is sensitive to electronic/structural shifts at sites remote from the core ring system, but still part of the conjugated system.

These data show that FSFs are extraordinarily sensitive to subtle changes in chemical structure and point to the rationale for sensitivity towards different SCRAs observed previously [7]. More specifically, it envisages shifts in the distribution of conformational states and consequently electronic structures. Therefore, shifts in cross-conjugation may be the drivers of the observed differences in the FSFs. This is considered in detail below.

### (ii) UV Irradiation Produces a Molecule Specific FSF

Observations of FSF measurements taken over extended time periods suggest that degradation of the study compounds occurs under UV irradiation. Indeed, indoles and indazoles are known to be photochemically reactive on UV irradiation [11]. Accordingly, arrangements explore the potential for tracking photochemical reactivity as an additional discriminatory probe of SCRAs and SCRA homologues.

FIG. 9A-E illustrates FSFs for (FIG. 9A) MDMB-4en-PICA, (FIG. 9B) MDMB-4en-PINACA, (FIG. 9C) MDMB-FUBICA, (FIG. 9D) MDMB-FUBINACA and (FIG. 9E) BZO-HEXOXIZID (also known as MDA-19) shown prior and post degradation; Red colouration represents emission with a relative intensity of one, and blue represents an intensity of zero. Inserts for (FIG. 9D) are coloured 0 to 0.025 relative intensity (blue to red) to highlight the minor changes observed. Also shown is the Prior-Post difference heat map. Conditions: 250 ng/ml in methanol, 20° C., 2 hours of irradiation with 300 nm LED. In particular, the FSFs are shown before and after 2 hours of irradiation (irradiation at 300 nm, corresponding to a peak in the absorption spectra as discussed below; continuous wave irradiation ˜0.2 mW). Corresponding pre-/post-irradiation difference maps are shown in FIG. 9A-E.

These SCRA compounds contain a range of structural groups including indole, indazole and oxoindole cores, and amino acid-derived linked groups. All five compounds are affected by UV irradiation, with the evolution of new products evident in all five difference maps. Although the majority of pre-degradation FSFs appear remarkably similar (with exceptions such as BZO-HEXOXIZID), the post-degradation FSFs are highly distinct, indicating potential for discrimination following degradation. For example, despite the similarity of the pre-degradation FSF, completely different post-degradation products for different ‘tails’ e.g., MDMB-4en-PINACA and MDMB-FUBINACA, can be seen in FIG. 9B and FIG. 9D, respectively. In addition, some SCRAs like BZO-HEXOXIZID (FIG. 9E) show minimal features in their initial/prior FSF, but can be easily observed post-irradiation.

The model series of SCRA analogues is used to study the observed degradation in detail.

FIG. 3A-C illustrates a time series of absorbance scans of compound 1d as it was degraded (FIG. 3A), a plot comparing the initial and final absorbance spectra (FIG. 3B), the changes in absorbance at wavelengths of interest (FIG. 3C); FIG. 3D-F illustrate a time series of absorbance scans of compound 2d as it was degraded (FIG. 3D), a plot comparing the initial and final absorbance spectra (FIG. 3E), the changes in absorbance at wavelengths of interest (FIG. 3F); FIG. 3G-I, show FSFs for 1d collected prior to sample irradiation (FIG. 3G) and post degradation (FIG. 3H), red colouration represents emission with a relative intensity of one, blue an intensity of zero. A heat map (I) showing the differences in emission intensity.

FIG. 3A-H shows the effect of UV irradiation as a function of time on the absorption spectra with respect to time upon irradiation for compound 1d (FIG. 3A-C) and compound 2d (FIG. 3D-F). Similar spectral changes for compounds 1a-c and 2a-c are observable. For compounds 1a-d, prior to irradiation, the spectra have defined absorption peaks at λ˜310 nm, ˜265 nm and ˜245 nm. FIG. 3B shows the difference absorption spectrum pre- and post-irradiation. From this, the spectral changes for compound 1d can be defined by absorbance changes at 6 defined wavelengths. FIG. 3C shows the time dependence of the spectral changes for compound 1d at these wavelengths. For each of these spectral features, the rate can be defined adequately by a single-exponential function:

$$Δ A = A exp ( - kt ) \qquad [ Equation 2 ]$$

Where:
A is the amplitude and
k is the rate constant obtained from time-dependent absorption change trace and
ΔA is the total absorbance change.

That these data can be adequately fit with a single exponential function is suggestive of a single (photochemical) process. The negative amplitude associated with the 310 nm peak convolves nearby peaks with an associated kinetic component, but the extracted rate constants are effectively the same. Moreover, these spectral changes all proceed with a similar rate constant (average k=0.18±0.07 (SD) s⁻¹ for 1d, suggesting that the absorption changes are reflecting the same rate-limiting process.

The data for 2d are similar for 1d, in that irradiation causes a gain of a similar new spectral feature (see FIGS. 3D and 3E), but red shifted by ˜10 nm compared to 1d.

However, from FIG. 3F, the rate constant for the observed shifts on irradiation are ˜50 times faster for 2d versus 1d, (k=8.6±2.9 (SD) s⁻¹ for 2d) and this trend is similar for the remaining members of the halogen series (1 and 2), discussed below. These data show that the presence of an ethanone-linker gives a dramatically increased rate of photochemical activity to produce a similar product. Moreover, for compound series 1 and 2, the kinetics are similar for the fluoro- and chloro-analogues, and an order of magnitude larger (and similar) for the bromo and iodo-analogues (FIG. 10). That is, a trend of increasing photochemical sensitivity with increasing electronegativity of the halogen group is observable.

Whilst the data does not definitively identify kinetically distinct species because a range of photochemical products is expected, it is possible to at least select for a similar ‘end-point’ of a photochemical step(s) with respect to time as a distinct exponential phase. For example, in the case of 1d, at ˜1000 min and for 2d at ˜30 min, given the irradiance of the light source used. FIGS. 3G and 3H show FSFs collected before and after the irradiation of 1d, respectively. FIG. 3I maps the differences in the relative intensities of the features present in the EEM of 1d. Equivalent plots are shown in FIG. 11A-L for compounds 2a-d.

Fitting these data to Eq 1 shows a single major species present in both the pre- and post-degradation spectra of 1d. These species are distinct (λₑₓ˜281 nm; λₑₘ˜346 nm and λₑₓ˜296 nm; λₑₘ˜404 nm). That is, irradiation of 1d to the kinetic endpoint, causes an effective complete loss of the parent fluorophore with formation of a single distinct fluorescent species. Moreover, the observed changes suggest mechanistic information on the photochemical breakdown. That is, the emergence of a new distinct fluorophore (FIG. 3H) suggests that the photochemical reaction mechanism involves the production of a new fluorescent species, or a shift in equilibrium of a specific electronic structure. Indeed, each of 1a-d give a distinct shift in the FSF on irradiation (FIG. 11A-L). In particular, FIG. 11A-L shows FSFs for compound 2a (FIG. 11A-C), compound 2b for (FIG. 11D-F), compound 2c for (FIG. 11G-I) and compound 2d (FIG. 11J-L) those FSFs shown were collected pre and post sample photodegradation. Red coloration represents emission with a relative intensity of one, blue an intensity of zero, and a heat map showing the differences in emission intensity;

These data are evidence that even highly structurally similar SCRA analogues can be discriminated based on photochemical reactivity, tractably monitored via shifts in their FSF.

### Computational Modelling

The data suggests that subtle shifts in the degree of cross conjugation (through varying linker length), and electro-negativity at sites separate from the fluorophore, is sufficient to measurably alter the molecular FSFs and photochemical reactivity. To investigate the origin of this sensitivity and the photochemical reactivity, a range of in silico methods were used. Calculations were performed on compounds, 1a-d and 2a-d at the B3LYP-D3(BJ)/def2SVP level of theory. All calculations were performed under the integral equation formalism polarizable continuum model (IEF-PCM) solvation model for methanol. Due to the flexible nature of the linker groups, conformational searches were performed for each molecule using the OPLS3e force field in Schrodinger's Macromodel (Ver. 12.6) and the resultant conformers were taken forward to DFT.

Optimisations were performed in Gaussian 16 (Rev. A.03) [13] and the quasiharmonic free energies were obtained at a constant temperature of 298.15 K and a concentration of 1 moldm⁻³ [14].

Table S2 below shows a range of data obtained for 1a-d and 2a-d, including the Boltzmann weighting of each conformer. Since cross-conjugation was suspected to influence the FSFs produced, the planarity of two ring systems either side of the linker group in compounds 1a-d was investigated by measuring the dihedral angle over carbon-3, -10, -11, and -16 (Table S2).

### Table S2.

Data calculated via DFT calculations for the lowest energy conformers of compounds 1a-d and 2a-d. Quasiharmonic energies have been calculated at a temperature of 298.15 K and a concentration of 1 moldm⁻³. The Boltzmann weighting of each conformer in the population are shown, with the distance between the halogen atom and the hydrogen atom on carbon-2, and the angle between the two planar ring systems (for compounds 1a-d). The asterisk pattern shows the conformers with the lowest energy (*), second lowest energy (**), and smallest distance between C-2 hydrogen and halogen atom (***).

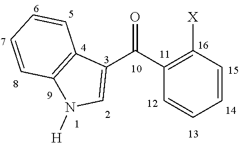

| | | | | | Distance | |
|---|---|---|---|---|---|---|
| | qh- | | | | between | |
| | G(T)_SPC/ | Qh-G(T)/ | | Boltzmann | H and X/ | Dihedral/ |
| Structure | hartree | kcal mol⁻¹ | | Weight | Angstrom | degrees |
| Fluoromethanone_1 | −806.759 | −506249 | | 0.609 | 2.590 | 49.9 |
| Fluoromethanone_2 | −806.758 | −506249 | | 0.305 | 4.592 | 130.0 |
| Fluoromethanone_3 | −806.757 | −506248 | | 0.066 | 4.692 | 48.5 |
| Fluoromethanone_4 | −806.756 | −506247 | | 0.019 | 5.399 | 132.6 |
| Chloromethanone_1 | −1167.071 | −732348 | | 0.815 | 3.263 | 68.2 |
| Chloromethanone_2 | −1167.069 | −732347 | | 0.185 | 4.859 | −67.6 |
| Bromomethanone_1 | −3281.958 | −2059460 | | 0.776 | 3.493 | 72.6 |
| Bromomethanone_12 | −3281.957 | −2059459 | | 0.224 | 4.904 | 71.1 |
| Iodomethanone_1 | −1005.578 | −631010 | (*) | 0.770 | 3.741 (***) | −74.7 |
| Iodomethanone_2 | −1005.577 | −631009 | | 0.230 | 4.983 | −73.6 |
| Fluoreoethanone_1 | −846.028 | −530890 | (*) | 0.580 | 2.306 (***) | — |
| Fluoreoethanone_2 | −846.024 | −530888 | | 0.020 | 6.732 | — |
| Fluoreoethanone_3 | −846.026 | −530889 | | 0.099 | 4.049 | — |
| Fluoreoethanone_4 | −846.027 | −530890 | | 0.273 | 5.032 | — |
| Fluoreoethanone_5 | −846.025 | −530889 | | 0.026 | 5.366 | — |
| Fluoreoethanone_6 | −846.022 | −530887 | | 0.002 | 3.936 | — |
| Chloroethanone_1 | −1206.340 | −756990 | (**) | 0.377 | 2.675 (***) | — |
| Chloroethanone_2 | −1206.340 | −756990 | (*) | 0.442 | 4.713 | — |
| Chloroethanone_3 | −1206.337 | −756989 | | 0.019 | 7.162 | — |
| Chloroethanone_4 | −1206.3339 | −756989 | | 0.112 | 4.241 | — |
| Chloroethanone_5 | −1206.337 | −756988 | | 0.033 | 5.479 | — |
| Chloroethanone_6 | −1206.337 | −756988 | | 0.013 | 6.067 | — |
| Chloroethanone_7 | −1206.336 | −756987 | | 0.005 | 5.415 | — |
| Chloroethanone_8 | −1206.334 | −756986 | | 0.001 | 3.899 | — |
| Bromoethanone_1 | −3320.142 | −2083420 | (*) | 0.545 | 2.832 (***) | — |
| Bromoethanone_2 | −3320.140 | −2083419 | | 0.036 | 7.322 | — |
| Bromoethanone_3 | −3320.141 | −2083420 | | 0.087 | 5.517 | — |
| Bromoethanone_4 | −3320.142 | −2083420 | | 0.204 | 4.347 | — |
| Bromoethanone_5 | −3320.140 | −2083420 | | 0.040 | 6.186 | — |
| Bromoethanone_6 | −3320.141 | −2083420 | | 0.087 | 5.517 | — |
| Bromoethanone_7 | −3320.136 | −2083417 | | 0.001 | 3.966 | — |
| Bromoethanone_8 | −3320.136 | −2083417 | | 0.001 | 3.967 | — |
| Iodoethanone_1 | −1044.064 | −655160 | (**) | 0.179 | 3.046 (***) | — |
| Iodoethanone_2 | −1044.061 | −655158 | | 0.015 | 7.522 | — |
| Iodoethanone_3 | −1044.063 | −655159 | | 0.052 | 5.580 | — |
| Iodoethanone_4 | −1044.063 | −655160 | | 0.088 | 4.459 | — |
| Iodoethanone_5 | −1044.065 | −655161 | (*) | 0.664 | 4.592 | — |

| | | | | | |
|---|---|---|---|---|---|
| Iodoethanone_6 | −1044.057 | −655156 | 0.000 | 4.029 | — |
| Iodoethanone_7 | −1044.057 | −655156 | 0.000 | 4.029 | — |

From Table S2 and Scheme 3 set out in FIG. 13A it can be found that the two ring systems become increasingly perpendicular to one another as the halogen size increases, with compound 1d having the largest dihedral angle for both conformers. This observation can be attributed to the increased steric bulk of the larger substituted iodine atom. In all but two compounds, it is noted that the halogen atom is situated in closest proximity to the hydrogen atom bonded to carbon-2 in the lowest energy conformers (Table S2; ‘measured distance between halogen X and hydrogen H’).

Previous work suggests that irradiation of these compounds can cause degradation into conjugated ring structure 7, with the elimination of compound HX (Scheme 3 shown in FIG. 13A) [15]. The close proximity of these atoms in the modelled structures suggest that elimination of molecule HX would be possible with limited energy required for molecular rotation, lowering the energy barrier for elimination. Similarly, the elimination of compound HX from compound 2a-d would produce the aromatic four-ring compound 8 (Scheme 4 shown in FIG. 13A).

Different degradation pathways using DFT and TDDFT calculations were investigated. It is possible to optimise the most stable conformers of compounds 1c and 2c in the ground (S₀) and excited (S1, S2 and S3) electronic states. For these calculations, the cis and trans conformations (with respect to the position of C═O and N—H) at the (TD)-ωB97XD/6-311+G(d) level of theory were considered. The main processes relevant for this study are summarized in FIG. 13B-D.

In FIG. 4A-D, the assignment of the main transitions in the in the absorption and emission spectra of the brominated molecules 1c and 2c and their derivatives. Similar assignments can be done for the other halogenated systems. In both systems, the first three absorption bands are assigned to π-π*S₀→S_{2,3,4} transitions (FIG. 4A and FIG. 4B).

Transitions to S₁ in all the energetically accessible structures (ΔG≤0.06 eV) show very low probability due to its n-π* character (FIG. 13B-D). The predicted absorption energies are within a range of 0.2-0.5 eV with respect to the experimental values (FIG. 13B-D). Such overestimation is systematically observed in conjugated systems computed with long-range corrected functionals with the default value of the range-separation parameter γ (in this case γ=0.2 a₀⁻¹). This is because the HOMO energies tend to be lower, whereas the LUMO's higher conducting to a bigger HOMO-LUMO gap energy [17].

Provided with the excitation wavelengths used in this work, molecules can be excited up to S₃ (FIG. 4A and FIG. 4B), thus, radiative, reactive and nonradiative deactivation processes to lower states also play a role in the photochemistry. Herein the stability of the different species stabilized in the excited states is analysed. Based on calculations, the emission spectra of 1c and 2c were assigned to the electron transitions of different species (FIG. 4C and FIG. 4D).

There are at least four species that can contribute to the emission spectrum of 1c (FIG. 4C). Emission from the 1c cis species is unlikely as it originates from highly excited electronic states (Kasha's rule). The cis H⁺ transfer species are not accessible (FIG. 13B-D). Emission from 7 involves the state S₃, while the process is energetically favourable, relaxation to lower energy states will be faster. Therefore, fluorescence is expected to occur mostly from S₁→S₀ transition of the 1c trans species. There are at least six different species that could contribute to emission after of 2c (FIG. 4D). Nonetheless, in this case, the degradation products (molecule 8) seem to be the main contributor with the keto product the main one based on its greater stability and higher emission oscillator strength (FIG. 13C-D and FIG. 4D). In this case, fluorescence of 8 is predicted to occur from S₁, as it is expected from Kasha's rule. In fact, these are the only two species for which fluorescence from S₁ is predicted near the observed emission bands. Besides, degradation products are the most stable species in the excited state.

### Characterisation of Degradation Products

Initial investigation of these degradation systems confirmed the elimination of molecule HX during degradation as predicted in scheme 3 of FIG. 13A.

FIG. 5A-C illustrates FSFs for compound 7 (0.2, 0.05 and 0.025 mg/mL, respectively). Red colouration represents emission with a relative intensity of one, and blue represents an intensity of zero. Conditions: Samples in methanol, 20° C., 1 cm path length. Data has not had methanol blank subtracted due to uneven quenching of methanol signal across excitation wavelength and sample concentration ranges.

Further analysis of the degradation material by mass spectrometry confirmed the presence of 7 in the post-degradation mixture of 1d, and 8 in the degradation mixture of 2d. To further confirm the degradation product of 1d, compound 7 was successfully synthesized. By taking FSFs of 7 at 0.2, 0.05 and 0.025 mg/mL (as shown in FIG. 5A-C), it is evident that the fluorescent nature of this compound is concentration dependent. At a concentration of 0.2 mg/mL, a spectral feature can be identified at λ_{Ex}^{max}≈380 nm and λ_{Em}^{max}≈500 nm. This spectral feature can also be recognized in the difference map for the degradation of 1d (FIG. 3I), suggesting the evolution of 7 during degradation, amongst other products.

### The Potential for Photochemical Detection in Saliva.

SCRA use cannot always be inferred from possession. It has been shown that SCRA FSFs can be distinguished in Saliva, which suggested utility in detecting SCRA use from oral fluid samples. Given the excellent discriminatory potential of UV degradation described above, enhanced SCRA discrimination via UV irradiation is investigated for samples present in oral fluid.

Compounds 1d and 2d are used as exemplars owing to these molecules showing the most rapid rate of photochemical degradation.

FIG. 6A-H shows the change in absorption for saliva alone (FIG. 6A) and saliva with the addition of compound 2d (FIG. 6B). The major absorption band present at ˜289 nm for saliva is attributable to the high concentration of protein present in human saliva and is typical [18]. On addition of compound 2d (1 μg/ml) to saliva the major absorption band appears blue shifted with a maximum at 285 nm. On irradiation, the absorption of the saliva control sample shows little variance with respect to time. However, on addition of 2d (FIG. 6B), the data show a time-dependent blue-shift of the major absorption band to an absorption maximum of ˜279 nm, over 240 minutes. These data suggest that there are photochemically driven changes to chromophores (presumably 2d) that can be captured by absorption in a saliva matrix, but not that are observable in saliva alone. These data are then a positive indicator that FSFs might be able to capture the fluorescence signatures of photochemical degradation of SCRAs in saliva.

FIG. 6C-E shows the FSFs for saliva (FIG. 6C), saliva containing compound 1d (FIG. 6D), and saliva containing compound 2d (FIG. 6E). FIG. 6F-H show the difference maps after irradiation for 30 minutes. These data show a loss in emission attributable to protein aromatic amino acids at λ_{Em}≈350 nm for the saliva only sample (FIG. 6F), potentially reflecting degradation of these chromophores, which is as expected from an abundance of photochemical degradation studies on proteins [19][20][21][22]. Conversely, the presence of both compound 1d and compound 2d show an increase in emission around λ_{Em}≈350 (FIG. 6G-6H) accompanied by a diffuse loss in emission elsewhere in the FSF.

A range of concentrations have been reported for relevant molecular concentrations in saliva post-smoking, including a maximum of 22370 μg/L for THC²³ and 35 μg/L for JWH-018 [24]. Given the huge potential range of biologically meaningful concentrations we have opted to use ˜1 μg ml⁻¹ above.

FIG. 12A-H shows the FSFs for MDMB-4en-PINACA in saliva/methanol at concentrations of 10, 50 and 250 ng/mL. Variations observed in the 330-400 nm excitation region are likely due to differences in diet preceding saliva collection on those days. The potential for convolving species from diet/other consumption is noted. However, FSFs arising from human saliva are remarkably consistent, dominated almost entirely by the peak at λ_{Em}≈280 nm [7]. While detection at a concentration of 50 ng/ml produces a clear fingerprint, at 10 ng/ml the SCRA signal is obscured. This can be overcome by subtracting the specific saliva sample used on that day, indicating the potential for optimization of the data subtraction method for increased sensitivity. These data show the potential for SCRA detection below 50 ng/ml in saliva/methanol solution.

These data show the potential of photochemical degradation combined with FSF detection for SCRA analogues at a physiologically anticipated concentration. Given these findings, whether combusted SCRA (mimicking the effects of smoking SCRA material) could be similarly detected from a saliva-only control was explored. A smoking simulator for generating realistic combusted material is known [25]. For the purposes of this study combusted AM-694 was generated, AM-694 being the SCRA on which the analogues used in this study are based.

FIG. 7 shows the FSF difference map of pre- and post-irradiated combusted AM-694 in saliva (as in FIG. 6A-H). This difference map shows spectral changes on irradiation similar to the analogues discussed above in relation to FIG. 6F-H, with an increase in emission located around λ_{Em}≈350 nm and a diffuse decrease in emission across the rest of the FSF.

The decrease in emission (blue coloration in FIG. 7) is highly reminiscent of the FSF of combusted AM-694 recorded previously [7] which suggests, similar to the photochemical studies above, that irradiation leads to the loss of the parent SCRA FSF. Combined with the model series data, these data imply that the presence of SCRA in saliva ‘protects’ the emission centered at λ_{Em}≈350 nm, versus in the absence of the SCRA. This might be a definable characteristic of the presence of SCRA in saliva.

### Conclusions

The vast majority of SCRAs are built on a similar scaffold, with a high-quantum yield fluorophore at the ‘core’ position. This is based on the ready availability of indole/indazole precursor material and the chemical tractability of chemical substitution. The sensitivity of indole/indazole fluorescence has previously been used to show that excitation-emission matrices (FSFs in the manuscript) of these compounds are both distinctive of SCRAs and also of different SCRAs. The work described herein highlights that this sensitivity arises not just due to immediate substituents on the chromophore, but also at positions remote from the chromophore. Moreover, DFT calculations suggest this arises from a shift in the distribution of conformational and the excited states (electronic, emissive states) that each SCRA can access.

Data shows that, at least for SCRAs, FSFs are a powerful analytical detection methodology. However, for application in the field, one requires extreme sensitivity and robustness of detection, not least because detection of SCRAs has profound legal and social consequences. Combining the FSF detection approach with monitoring of the photochemical reactivity of SCRAs was considered. It has been found that photochemical discrimination is specific for individual SCRA analogues and that this can even be achieved from street material in saliva. This hybrid approach, which distinguishes the SCRA from a pre- and post-irradiation FSF difference map (such as FIG. 6A-H), is termed “photochemical fingerprinting”.

Photochemical fingerprinting has the advantage that it can be readily incorporated into a portable detection system. It is possible to construct portable fluorimeters built using UV LEDs as the excitation source. Recent advances in LEDs in this spectral region (<400 nm) mean they are bright (˜mW tunable), stable (thousands of hours) and have low spectral bandwidths (˜12 nm). FIG. 8A-C shows a portable device built for point-of-care SCRA detection via FSFs, which may be adapted to irradiate the sample with, for example, one of the LEDs (300 nm in this case) to drive photochemical degradation. With bright enough or multiple LED sources, a photochemical fingerprint could be rapidly produced at a range of different excitation wavelengths, further enhancing the detection potential. Moreover, as has been shown with BZO-HEXOXIZID, this approach could enable FSF detection with molecules that, prior to irradiation, have low quantum yields, expanding the detection scope to other drugs of abuse.

### Materials and Methods

All glassware was flame-dried under vacuum and all moisture sensitive reactions and reagent transfers were carried out under nitrogen.

### Synthesis of Compounds 1a-d

In a 250 mL two-necked round-bottomed flask, indole 3 (4.27 mmol) was dissolved in dry DCM (42.5 mL) under an inert atmosphere. After cooling in an ice bath for 10 mins, Et₂AlCl (1 mol/L in hexane, 6.4 mL) was syringed into the flask via slow, dropwise addition. The mixture was stirred in the ice bath for 30 mins before the slow dropwise addition of the corresponding acyl halide 4a-d (6.4 mmol) diluted in 10 mL of DCM. The resulting mixture was stirred at room temperature overnight (16 h), and then quenched with 20 mL sat. aq. NH₄Cl.

An off-white suspended solid formed in the reaction mixture due to aluminium salts precipitating out of solution upon quenching. This was gravity filtered to produce a clear filtrate that was extracted with additional DCM (2×35 mL). The aqueous layer containing the majority of the aluminium salts was discarded. The DCM layer was washed with distilled water (3×25 mL), dried (MgSO₄), and evaporated to afford the crude product. This was purified using silica gel column chromatography (petroleum ether:ethyl acetate 4:1, R_{f}=0.18) to produce 1a-d, which was characterized with NMR and IR spectroscopy, melting point, and mass spectrometry (see Supporting Information).

Synthesis of acetyl chloride 6a-d

A mixture of 2-halogenated acetic acid 5a-d (10 mmol) and SOCl₂ (25 mL) was stirred at 100° C. for 3 hours, under reflux. 10 mL toluene was added and any excess SOCl₂ was removed via distillation at 105° C. Toluene was then removed under reduced pressure to afford 2-halogenated acetyl chloride 6a-d.

### Synthesis of Compounds 2a-d

The same synthesis method, as was used for compounds 1a-d, was undertaken using the corresponding phenylacetyl chloride 6a-d. However, the crude product was instead purified via trituration with ethyl acetate, then recrystallization from chloroform to give 2a-d. This was characterized with NMR and IR spectroscopy, melting point, and mass spectrometry (see Supporting Information).

### Computational Study

To account for molecular flexibility, comprehensive conformational searches were performed for all eight compounds (1a-d and 2a-d) using Schrödinger's MacroModel (Ver 11.3) [12]. The OPLS3e force field and PRCG minimisation method were chosen for conformational searches, and a mixed torsional/low-mode sampling approach was adopted. All structures were further optimised using DFT, with geometry optimizations being performed in Gaussian 16 (Rev. A.03) [13]. Calculations were completed at the B3LYP-D3(BJ)/def2svp level of theory. Grimme's D3 dispersion correction with Becke-Johnson damping was included to better account for weak intermolecular interactions, as previously utilised in the literature. Implicit solvation using IEF-PCM was included in all calculations, with methanol as the chosen solvent (dielectric constant ε=32.613). The temperature (298 K) and concentration (1 mol dm−3) corrected quasiharmonic free energy of each conformation was obtained using the GoodVibes [14].

Excited states calculations were performed for the most stable conformers of 1c, 2c and AM-694 molecules. We consider the molecules with conformations cis and trans with respect to the positions of the carbonyl and the amine groups (conformers Bromomethanone_1, Bromomethanone_2, Bromoethanone_1, Bromoethanone_2 and AM-694_1, see Table S2). The optimizations of ground and the S₁, S₂, S₃ states were performed at the TD-ωB97XD/6-311+G(d) level of theory in methanol with the IEFPCM model. Every stable geometry was tested as a true minimum by a vibrational frequencies analysis obtaining zero imaginary frequencies. The thermodynamic functions ΔH, ΔS and ΔG were computed for every sable geometry obtained at 298 K within the harmonic oscillator and rigid rotor approximations as implemented in Gaussian 16 [13]. The absorption and emission energies were computed as the vertical transition from the equilibrium structure of the electronic state from which the transition occurs.

### Absorption and Fluorescence Spectra and Photo-Degradation

Fluorescence readings were collected using a PerkinElmer LS50B luminescence spectrometer (PerkinElmer, Waltham, MA, USA) with attached water bath for temperature regulation. Sample and background measurements were taken at 20° C. The excitation and emission slit widths were varied between 2.5 and 12 nm depending on the signal. For each measurement, a corresponding background reading was directly subtracted, particularly to remove contributions from Raman scattering. The EEMs shown have had the signal contributions from excitation light and second order scattering removed.

Absorbance measurements were taken using a Varian Cary 50 Scan UV-VIS Photometer. Absorbance was measured from 800 nm to 200 nm at 1 nm intervals with a scan rate of 600 nm/min.

Sample degradation was carried out using a M300L4-300 nm, 26 mW Thor Labs LED. The LED was in a fixed position relative to the cuvette holder used in the irradiation step keeping the intensity of the light delivered consistent. Samples were fully contained during degradation, so volume and sample concentration were unchanged. The synthetic strategy and purification process of these samples is described above. Samples were dissolved in HPLC methanol >99.9% purity (Sigma-Aldrich, St Louis, MO, USA.

Oral fluid samples were collected from volunteers who confirmed no legal or illegal drug use in the preceding month. Saliva samples were centrifuged for 15 minutes at 4° C., to separate solid material, before being passed through a 0.44 μm syringe-driven filter.

The data set out above show that FSFs are extraordinarily sensitive to subtle changes in chemical structure and point to the rationale for sensitivity towards different SCRAs observed previously. More specifically, it envisages shifts in the distribution of conformational states and consequently electronic structures. Therefore, shifts in cross-conjugation may be the drivers of the observed differences in the FSFs. This is considered in detail below. In particular, the data of above relates to SCRA compounds containing a range of structural groups including indole, indazole and oxoindole cores, and amino acid-derived linked groups. All five compounds studied were affected by UV irradiation, with the evolution of new products evident in all five FSF difference (post vs pre photochemical change) maps. Although the majority of pre-degradation FSFs for the SCRAs under study appeared remarkably similar (with exceptions such as BZO-HEXOXIZID), the post-degradation FSFs are highly distinct, indicating potential for discrimination of SCRA following photochemical degradation.

### Applicability of Enhanced Fluorescence Spectral Fingerprinting (FSF) to Non-SCRA Substance Detection

Aspects described recognise that the hybrid FSF and photochemical fingerprinting approach described in relation to SCRA detection above may have applicability to test substances other than those in solution and/or to substances other than SCRA compounds.

Many aromatic molecules are photochemically active, meaning either they rapidly (relatively) degrade in the presence of specific wavelengths of irradiation and/or that they undergo photochemical reactions with, for example, a solvent in which they are placed, themselves, or other molecules of the same species. Those photochemical reactions may give new photoproducts that have altered absorption/emission fluorescence spectra. As a result, a hybrid fluorescence fingerprinting and photochemical transition approach may offer a route to detection of some aromatic molecules.

In particular, aspects described recognise that monitoring changes in an excitation-emission matrix (spectral fingerprint; FSF) of a molecule, in combination with photochemical degradation, allows discrimination of certain molecules. In other words, an aromatic molecule may have an associated “hybrid photochemical fingerprint”. The fingerprint according to described aspects comprises a signal attributable to fluorescence emission from a test substance.

It is not a priori possible, at least to a reasonable level of tractability, to predict the photochemical products of specific molecules and how this will affect their emission/absorption spectra in a given solvent or solid environment. For clarity, such in silico calculations are possible, but do not yield full excitation emission maps, or in a manner that is timely and therefore is not routinely tractable.

It is therefore difficult to predict which aromatic molecules are candidates for detection via the methodology described in detail above.

The inventors have recognised that the hybrid fluorescence photochemical fingerprinting methodology supports the discrimination of a range of illicit drugs from the following classes: cannabinoid (including, of course, the synthetic cannabinoid compounds described above); opioid (including synthetic opioids); benzodiazepine; cathinone (mephedrone); steroid (CDMT) and sedative (xylazine).

### Methodology

Intrinsic fluorescence spectroscopy (IFS) does not require artificial probes since it detects fluorophores which exist as natural components of an analyte material. Approaches described recognise that individual aromatic compounds may have a naturally occurring characteristic excitation and emission fluorescence spectra. A two-dimensional excitation/emission matrix (EEM; FSF as above) therefore could contain a detailed report on the chemical composition of a compound under test. The discriminatory ability of fluorescence, for those compounds which have a fluorescence response, is therefore potentially extremely high. Coupled with this, many aromatic molecules exhibit a photochemical response, as described above. That photochemical response may result in a detectable change in a fluorescence response of a compound and therefore the combination of a fluorescence and photochemical fingerprinting methodology may have significant benefits.

A general methodology for performing a hybrid fluorescence photochemical fingerprinting method according to aspects may comprise the following steps:
Step 1: Irradiation of a sample under test, sequentially across a plurality of excitation wavelengths. In one example methodology, the plurality of excitation wavelengths comprise: 255 nm, 265 nm, 275 nm, 285 nm, 295 nm, 305 nm, 320 nm, 340 nm, 365 nm, 375 nm, 385 nm, 395 nm and 405 nm. It will be appreciated that a difference plurality of excitation wavelengths may be selected. Such excitation wavelengths may be selected such that they are spaced at similar reasonable intervals to achieve coverage of a desired excitation spectral range.
Step 2: Collection of emission from the sample under test at each of the excitation wavelengths. That collection may comprise a full spectral acquisition, for example, using a monochromated spectrometer.
Step 3: Irradiation of the sample under test at a wavelength or wavelengths determined to be likely to induce a photochemical change in the sample under test. That wavelength, or wavelengths may, for example, be selected to be at, or near to, an absorption maximum of the sample under test (or maxima, or as a combination of wavelengths that produce a change in the spectral fingerprint). The irradiation intended to induce a photochemical change may occur for a period of time and/or an intensity selected to induce a measurable change in a spectral fingerprint obtained via step 2. It will be appreciated that various methods to determine appropriate irradiation likely to induce photochemical change, for those substances in a sample likely to be subject to such a change, can be implemented. By way of general example:

Determining a wavelength for irradiation of the sample to induce a photochemical change in the provided sample may comprise determining a wavelength of an absorption maxima of the provided sample and irradiating the provided sample with irradiation having a wavelength close to the determined absorption maxima to induce a photochemical change in the provided sample.

Determining a wavelength for irradiation of the sample to induce a photochemical change in the provided sample may comprise: measuring an absorption spectrum of a plurality of drugs and evaluating the measured absorption spectra to select one or more wavelength for irradiation of the provided sample. The evaluation may, for example, comprise selecting a range of wavelengths for irradiation, that range being selected to encompass the wavelengths determined to induce a change in the drugs in the library.

The wavelength for irradiation may comprise a wavelength, a plurality of wavelengths, or range of wavelengths in the ultraviolet region of the spectrum. The wavelength for irradiation of the provided sample may comprise a wavelength, or plurality of wavelengths, in the range from around 230 nm to around 400 nm.

In some implementations, for example, a provided sample may be irradiated with broadband UV, or cycle different UV wavelengths. Such an approach may induce a photochemical change across a large range of drugs in a library and therefore result in a reasonable change that a photochemical change will be induced if a drug of interest is present in a provided sample.
Step 4: Repetition of steps 1 and 2 to enable collection of emission from the photochemically changed sample under test as full spectral acquisition, for example using a monochromated spectrometer.
Step 5: Comparison of the fingerprints obtained in Step 2 and Step 4. In some implementations, the comparison may comprise creation of a difference map between the fingerprints obtained in steps 2 and 4 to give the hybrid photochemical fingerprint of the sample under test.
Step 6: Evaluation of one or more of the fingerprints or comparison of fingerprints obtained in steps 2, 4, or 5 to identify the sample under test. In some implementations, the evaluation may comprise comparison to a library of hybrid photochemical fingerprints of known substances. The comparison may comprise looking for identity or matching, a match within a threshold or similar and may occur by means of statistical analysis, including machine learning approaches.

It will be appreciated that changes in fingerprint may occur in both the ultra-violet and visible spectral regions. Furthermore, assessment of photochemical fingerprints might be conducted visually, where there is, for example, an obvious (visible to the human eye, for example) colour change in or on the provided sample.

The hybrid photochemical fingerprinting methodology described captures and includes information on both new fluorescent species that emerge as a result of photochemical reactions but also the loss of fluorescent/absorptive species as they are photochemically degraded. Both of these physical changes are reflective of the specific chemistry of the analyte.

### Apparatus

FIG. 14A-E illustrate various aspects of an apparatus configured to perform the hybrid photochemical fingerprinting method described generally above.

FIG. 14A illustrates schematically some main components of an apparatus configured to perform the hybrid photochemical fingerprinting method described generally above. The apparatus 1 comprises: an LED ring 10 configurable to irradiate a sample (not shown in FIG. 14A) and a spectrometer 20 configurable to collect emission from the sample. The apparatus 1 further comprises a microcomputer 30, configured to control operation of the LED ring 10 via LED drivers 40, and to control operation of the spectrometer 20. The microcomputer 30 may comprise reconfigurable storage. That storage may be configured to store, temporarily or otherwise, data generated by the spectrometer and/or the LED drivers. The storage may be coupled with a processor, configured to process and evaluate the data generated by the spectrometer and/or the LED drivers. The storage may comprise a locally held library of hybrid photochemical fingerprints associated with a range of substances. In an alternative implementation, the apparatus may comprise communication circuitry, configured to communicate with a remote library of hybrid photochemical fingerprints associated with a range of substances.

The apparatus of FIG. 14A further comprises an operation button 50 in communication with the microcomputer to initiate various phases of the photochemical fingerprinting method, a display 60 to allow a user to interface with the apparatus 1 and receive information about the photochemical fingerprinting method and the result of that method in relation to a sample under test.

The apparatus of FIG. 14A also comprises a temperature sensor 70, in communication with the microcomputer 30. Knowing the ambient temperature, or the temperature of a sample under test may allow an assessment to be made regarding, for example, irradiation duration for the purposes of induction of a photochemical change in a sample under test.

The apparatus of FIG. 14A also comprises an accelerometer 80, in communication with the microcomputer 30. The signal from the accelerometer may be used to determine whether the apparatus and/or sample are moving and therefore potentially whether an obtained set of results are likely to be valid.

FIG. 14B illustrates an LED ring 10 for use in an apparatus according to an aspect. The LED ring of FIG. 14A shows 12 equally spaced LEDs 12 mounted on a concave annular base 14 such that each LED is positioned to irradiate a sample placed towards the central portion of the annulus. In the LED ring of FIG. 14B, the LEDs are in a 1-inch diameter ring arrangement, equally circumferentially spaced. An aperture 16 is provided in the centre of the annular base 14.

FIG. 14C-D illustrate a spectrometer 20 and LED ring 10 for use in an apparatus according to an aspect. FIG. 14C shows the spectrometer 20 in a custom housing mountable on, and connectable to, the LED ring 10. A collimating lens 25 is provided in the front of the spectrometer in the region of the aperture 16 of the LED ring 10. The entrance of the collimating lens 25 in the apparatus shown is arranged such that it is locatable 15 mm from the sample surface. FIG. 14D is a side view showing that the entrance to the spectrometer 20, via which emission from a sample is collected, is aligned centrally with the aperture 16 provided in the LED ring 10.

FIG. 14E illustrates a complete device 1 showing an outer housing 100 in which the LED ring 10, spectrometer 20, microprocessor 30 and other components are located. On the outside of the housing 100 the multifunctional buttons 50 and the display 60 can be seen. FIG. 14E also shows a sample holder 200 for liquid samples. The sample holder 200 comprises a central sample well 210. In the implementation shown, the central sample well 210 comprises black HDPE, with a custom machined well which is 1 inch wide, 10 mm deep and of a true hemispherical construction. The sample holder 200 further comprises an alignment guide 220, in this case, in the form of a shaped protrusion configured to abut a side of housing 100 when the apparatus 1 is aligned and in a position to test a sample located in the sample well 210. The geometry and arrangement of the LEDs of the LED ring relative to the entrance slit of the spectrometer 20 can significantly impact the resultant spectral fingerprints and the photochemical changes/degradation of the samples. Shown here is one possible implementation.

The sample holder 200 is similarly important for the resultant spectral fingerprints. That is to say, the material and shape of the sample holder may affect the resultant spectral fingerprints. The implementation shown and described is one possible implementation having geometry optimised as described above. The shaping, configuration and design, together with material selection mitigates possible photochemical damage to the sample holder that would otherwise affect the resultant spectral fingerprints, as well as balancing the signal intensity and tractability of use in a practical setting (including, for example, cleaning between samples).

Photochemical Fingerprinting for Substances Other than Synthetic Cannabinoids

### Tetrahydrocannabinol (THC)

Cannabidiol (CBD) is a generally legal substance; tetrahydrocannabinol (THC) is a psychoactive substance which is generally not legal. CBD products should not contain THC. The disclosure set out above is confined to the study of SCRAs. In fact, it details a study of a series of SCRA model systems (which mimic a specific SCRA known as AM-694). The spectrochemical properties of molecular systems are highly dependent on their structure. The SCRA model systems feature electronegative halogen substituents. The SCRA model systems also feature an electronegative indole N-atom. Both of these structural features contribute significantly to the observed fluorescence properties and degradation of these model systems, but are crucially absent in the THC molecule. There is no disclosure or teaching above which would result in the person skilled in the art inferring that the THC molecule would be expected to exhibit a similar fluorescence sensitivity and would exhibit characteristic degradation of the type that would enable it to be fingerprinted in the way described. Nonetheless, it has been determined that a photochemical fingerprinting methodology can be used to detect and identify the presence of THC in a solution.

FIG. 15A-C show graphically the emission spectra of CBD, THC and a combination irradiated over time with a 265 nm irradiation source to induce a photochemical change.

FIG. 15A-C shows the emission spectra of CBD, THC and the combination irradiated over time with a 265 nm source. Each subsequent spectrum represents a new time point of data collection (every 5 minutes for 30 minutes).

FIG. 15A shows how a solution of CBD irradiated at 265 nm, will show some simple photodegradation in the form of a decrease in fluorescence emission without changes in the structure of the emission band.

FIG. 15B shows how a solution of THC shows both photodegradation and new fluorescent species attributable to novel electronic structures (photoproducts), with new peaks at ˜360 and 380 nm, amongst others when exposed to similar irradiation.

FIG. 15C shows how, at a 10:1 ratio of CBD: THC, the new peaks from the photoproducts of THC are evident and allows discrimination of presence of THC in the presence of a high molar ratio of CBD.

It can therefore be understood that, if induction of a photochemical change is implemented, the photochemical fingerprinting methodology described above can be used to identify THC, but also THC in the presence of CBD. This results from the THC having a different photochemical activity compared to CBD, despite the structures being very similar.

### Other Molecules

It is not a simple task to predict whether a molecule is a candidate for detection via the fluorescence photochemical fingerprinting technique described above. It has, via experiment, been determined that the hybrid fluorescence photochemical fingerprinting approach above is valuable and important in detection of various substances of abuse, including, for example, opioid (including synthetic opioids); benzodiazepine; cathinone (mephedrone); steroid (CDMT) and sedative (xylazine).

FIG. 16A-F shows fluorescence spectral fingerprints (FSFs) obtained pre- and post-irradiation to induce a photochemical change, together with a difference map comparison between the FSFs for 6 different molecules (Flunitrazepam, Fentanyl, Mephedrone, Etonitizene, Dihydrocodiene and CDMT in each column left to right) tested in an ethanol solution.

FIG. 16A-F shows the spectral fingerprints for a range of molecules and showing their pre/post irradiation fingerprints and the difference map (hybrid photochemical fingerprint) according to various aspects. The examples given are in Ethanol and at a range of concentrations from 100 μg to 2 mg mL−1. The samples were irradiated with broad band UV for ˜5 minutes. The background colouration of each figure represents zero as the colour key at right. Note that the difference map data uses a +/−scale.

Some implementations recognise that approaches in accordance with aspects may be deployed in relation to molecules which do not have a significant photochemical response. In particular, approaches may be deployed in relation to compounds which have a minimal, but non-zero, photochemical response. A lack of photochemical degradation can itself be indicative of the chemical structure when, for example, combined with a knowledge of an initial spectral fingerprint.

FIG. 17A-E shows fluorescence spectral fingerprints (FSFs) obtained pre- and post-irradiation to induce a photochemical change, together with a difference map comparison between the FSFs for 5 different molecules (Clonazepam, Diazepam, Alprazolam, Etizolam, and Xylazine in each column left to right) tested in an ethanol solution. The examples given are in ethanol and at a range of concentrations from 100 μg to 2 mg mL−1. Irradiation with broad band UV for ˜ 5 minutes. The background colouration of each figure represents zero as the colour key at right. Note that the difference map data uses a +/−scale.

Accordingly, it is demonstrated that the hybrid photochemical fluorescence fingerprinting methodology can be used to detect a range of substances tested in solution.

### Extension of Hybrid Photochemical Fluorescence Fingerprinting to Solid Substances and Substances Adsorbed on a Physical Matrix

Aspects recognise that the approach described above in relation to samples tested in solution can be extended to situations where drugs are available in a solid, for example, powdered, or tablet form, or adsorbed onto a physical matrix (for example, paper).

FIG. 18A-D shows fluorescence spectral fingerprints (FSFs) obtained pre- and post-irradiation to induce a photochemical change, together with a difference map comparison between the FSFs for 4 different cases: no drug adsorbed onto a paper matrix; a synthetic cannabinoid adsorbed onto a paper matrix; a Benzodiazepine adsorbed onto a paper matrix; and a Nitazene adsorbed onto a paper matrix. Each row relates to one case. The spectral fingerprints of FIG. 18A-D for three different drugs on white paper are shown: pre (column at left) and post (middle column) irradiation with broad band ultraviolet light. The column at right shows the corresponding difference maps for each condition. Similar results are achieved if a drug is adsorbed onto yellow or black paper or card.

FIG. 19 shows a photograph of samples on paper after irradiation to induce a photochemical change. FIG. 19 gives a visual image of clobazam and etonitazene samples on white paper after irradiation/after being subject to testing using apparatus such as that shown in FIG. 14E, adapted so that the sample holder receives a paper sample, or adapted so that the apparatus 1 in housing 100 is abutted against a sample paper under test. The paper shown on the left of FIG. 19 shows a control where only solvent is added to paper, the middle paper shows paper on which clobazam was added and the right paper shows paper on which etonitazene added. In each case the drug samples are at 100 μg/cm².

It can be seen that the photochemical fingerprints (difference maps) vary in a drug-specific manner when the drug is adsorbed onto paper. The change in the fingerprints is reflected both in the change in the signature of the optical brightening agents of the paper itself (centered at ˜420 nm in FIG. 18A-B) but also the loss/gain of new spectral features which are attributable to photochemical degradation/reaction of the adsorbed drugs.

The changes in the optical brightening agents of the paper is assumed to be due to changes in resonance energy transfer/absorption of the emitted light from the optical brightening agents by the drugs and therefore varying as the drugs are photochemically degraded/reacting to give new products.

Visual changes are also clear in some cases (as shown in FIG. 19), where irradiation of the samples gives rise to a visual colour change in addition to the spectral fingerprint shown in FIG. 18A-D, which is rather more focused on the ultraviolet region.

In other words, although the optical brightening agents of paper change their fluorescence in the presence of drugs (see FIG. 18A), they also change characteristically as the photochemical fingerprint in the presence of drugs, thereby supporting use of the hybrid photochemical fingerprinting method directly in relation to drugs adsorbed onto a physical matrix, in this case, white paper.

Some aspects recognise that the sample under test may be a physical matrix onto which a substance of interest may be adsorbed. An appropriate library of reference photochemical fingerprints may be available to the apparatus and it may be possible for a user to select whether the fingerprinting methodology is being applied to a sample in solution or a sample on a physical matrix.

In relation to a solid drug form, for example, in the case that a provided sample is a powder or tablet, it is possible adapt the methodologies described herein. In particular, a provided powder may be placed in sample holder 200 and appropriate FSFs acquired pre- and post-photochemical change. An appropriate library of reference photochemical fingerprints may be available to the apparatus and it may be possible for a user to select whether the fingerprinting methodology is being applied to a sample in solution, a sample on a physical matrix or a solid sample.

FIG. 20 illustrates schematically some components of an apparatus according to some arrangements; and FIG. 21 illustrates schematically steps of methods performed in accordance with some arrangements.

FIG. 20 shows an apparatus 9000 for drug detection. The apparatus 9000 is configured to identify whether a drug is present in a provided sample. The apparatus comprises generally:
9010: circuitry configured to perform determining a first fluorescence spectral matrix associated with the provided sample by communicating with an excitation source and emission detector to effect: excitation of the provided sample at a first excitation wavelength; and reception of a fluorescence emission spectrum generated by the provided sample at the first excitation wavelength.

The circuitry 9010 being configured to effect repetition of the excitation and receiving steps across a range of excitation wavelengths which differ from the first excitation wavelength.
9020: circuitry configured to perform determining a wavelength for irradiation of the sample to induce a photochemical change in the provided sample and irradiating the provided sample with radiation having the determined wavelength. The irradiation may be performed by the excitation source or an alternative irradiation source. The excitation and irradiation sources may be substantially co-located within the apparatus 9000.
9030: circuitry configured to perform determining a second fluorescence spectral matrix associated with the provided sample by:
by communicating with an excitation source and emission detector to effect: excitation of the provided sample at a first excitation wavelength; and reception of a fluorescence emission spectrum generated by the provided sample at the first excitation wavelength. The circuitry 9030 being configured to effect repetition of the excitation and receiving steps across a range of excitation wavelengths which differ from the first excitation wavelength.
9040: circuitry configured to perform comparing the determined first and second fluorescence spectral matrix associated with the provided sample with a library of first and second fluorescence spectral matrices obtained from a plurality of drug samples.
9050: circuitry configured to perform triggering a positive identification of a drug in the provided sample if a match within a predetermined threshold is revealed by the comparison.

FIG. 21 illustrates schematically steps of methods performed in accordance with some arrangements. FIG. 21 shows a drug detection method 9100 to identify whether a drug is present in a provided sample, the method comprising:
9110: determining a first fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
9120: determining a wavelength for irradiation of the sample to induce a photochemical change in the provided sample and irradiating the provided sample with radiation having the determined wavelength;
9130: determining a second fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
9140: comparing the determined first and second fluorescence spectral matrix associated with the provided sample with a library of first and second fluorescence spectral matrices obtained from a plurality of drug samples; and
9150: if a match within a predetermined threshold is revealed by said comparison, triggering a positive identification of a drug in the provided sample.

A person of skill in the art would readily recognize that steps of various above-described methods can be performed by programmed computers. Herein, some embodiments are also intended to cover program storage devices, e.g., digital data storage media, which are machine or computer readable and encode machine-executable or computer-executable programs of instructions, wherein said instructions perform some or all of the steps of said above-described methods. The program storage devices may be, e.g., digital memories, magnetic storage media such as a magnetic disks and magnetic tapes, hard drives, or optically readable digital data storage media. The embodiments are also intended to cover computers programmed to perform said steps of the above-described methods. The term non-transitory as used herein, is a limitation of the medium itself (i.e., tangible, not a signal) as opposed to a limitation on data storage persistency (e.g. RAM vs ROM).

As used in this application, the term “circuitry” may refer to one or more or all of the following:
(a) hardware-only circuit implementations (such as implementations in only analog and/or digital circuitry) and
(b) combinations of hardware circuits and software, such as (as applicable):
   (i) a combination of analog and/or digital hardware circuit(s) with software/firmware and
   (ii) any portions of hardware processor(s) with software (including digital signal processor(s)), software, and memory(ies) that work together to cause an apparatus, such as a mobile phone or server, to perform various functions) and
(c) hardware circuit(s) and or processor(s), such as a microprocessor(s) or a portion of a microprocessor(s), that requires software (e.g., firmware) for operation, but the software may not be present when it is not needed for operation.

This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

Although example embodiments of the present invention have been described in the preceding paragraphs with reference to various examples, it should be appreciated that modifications to the examples given can be made without departing from the scope of the invention as claimed.

Features described in the preceding description may be used in combinations other than the combinations explicitly described.

Although functions have been described with reference to certain features, those functions may be performable by other features whether described or not.

Although features have been described with reference to certain embodiments, those features may also be present in other embodiments whether described or not.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

### REFERENCES

1 B. M. Amar, J. Ethnopharmacol., 2006, 105, 1-25.
2 European Monitoring Centre for Drugs and Drug Addiction, European Drug Report: Trends and Developments, Luxembourg, 2022.
3 A. J. Krotulski, A. Cannaert, C. Stove and B. K. Logan, Drug Test. Anal., 2021, 13, 427-438.
4 A. Alipour, P. B. Patel, Z. Shabbir and S. Gabrielson, Ment. Heal. Clin., 2019, 9, 93-99.
5 L. Mogler, M. Wilde, L. M. Huppertz, G. Weinfurtner, F. Franz and V. Auwärter, Drug Test. Anal., 2018, 10, 886-891.
6 A. Namera, M. Kawamura, A. Nakamoto, T. Saito and M. Nagao, Forensic Toxicol., 2015, 33, 175-194.
7 B. May, H. A. Naqi, J. A. Scott, I. S. Blagbrough, S. M. Husbands and C. R. Pudney, Anal. Chem., 2019, 91, 12971-12979.
8 A. J. Potts, C. Cano, S. H. L. Thomas and S. L. Hill, Clin. Toxicol., 2020, 58, 82-98.
9 R. M. Schelkun, A. J. Krotulski, D. M. lula and B. K. Logan, New Systematic Naming for Synthetic Cannabinoid “MDA-19” and Its Related Analogues: BZO-HEXOXIZID, 5F-BZO-POXIZID, and BZO-POXIZID, 2021.
10 X. Meng, T. Harricharran and L. J. Juszczak, Photochem. Photobiol., 2013, 89, 40-50.
11 E. M. L. Janssen, E. Marron and K. Mcneill, Environ. Sci. Process. Impacts, 2015, 17, 939.
12 Schrödinger Release 2021-4: MacroModel, Schrödinger, LLC, New York, NY, 2021.
13 M. J. Frisch, G. W. Trucks, H. B. Schlegel, G. E. Scuseria, M. A. Robb, J. R. Cheeseman, G. Scalmani, V. Barone, G. A. Petersson, H. Nakatsuji, X. Li, M. Caricato, A. V. Marenich, J. Bloino, B. G. Janesko, R. Gomperts, B. Mennucci, H. P. Hratchian, J. V. Ortiz, A. F. Izmaylov, J. L. Sonnenberg, D. Williams-Young, F. Ding, F. Lipparini, F. Egidi, J. Goings, B. Peng, A. Petrone, T. Henderson, D. Ranasinghe, V. G. Zakrzewski, J. Gao, N. Rega, G. Zheng, W. Liang, M. Hada, M. Ehara, K. Toyota, R. Fukuda, J. Hasegawa, M. Ishida, T. Nakajima, Y. Honda, O. Kitao, H. Nakai, T. Vreven, K. Throssell, J. Montgomery, J. A., J. E. Peralta, F. Ogliaro, M. J. Bearpark, J. J. Heyd, E. N. Brothers, K. N. Kudin, V. N. Staroverov, T. A. Keith, R. Kobayashi, J. Normand, K. Raghavachari, A. P. Rendell, J. C. Burant, S. S. Iyengar, J. Tomasi, M. Cossi, J. M. Millam, M. Klene, C. Adamo, R. Cammi, J. W. Ochterski, R. L. Martin, K. Morokuma, O. Farkas, J. B. Foresman and D. J. Fox, Gaussian 16, Revision A.03, Gaussian Inc., Wallingford CT, 2016.
14 G. Luchini, J. V. Alegre-Requena, I. Funes-Ardoiz and R. S. Paton, F1000Research, 2020, 9, 291.
15 W. Carruthers and N. Evans, J. Chem. Soc. Perkin Trans. 1, 1974, 1523-1525.
16 R. S. Bhatta, G. Pellicane and M. Tsige, Comput. Theor. Theory, 2015, 1070, 14-20.
17 C. Halsey-Moore, P. Jena and J. T. McLeskey Jr., Comput. Theor. Chem., 2019, 1162, 112506.
18 K. D. S. Nanda, K. Ranganathan, K. M. Umadevi and E. Joshua, J. Oral Maxillofac. Pathol., 2011, 15, 22-25.
19 C. Schöneich, J. Pharm. Pharmacol., 2018, 70, 655-665.
20 O. Mozziconacci and C. Schöneich, Adv. Drug Deliv. Rev., 2015, 93, 2-13.
21 G. Xue, K. Wang, D. Zhou, H. Zhong and Z. Pan, J. Am. Chem. Soc., 2019, 141, 18370-18374.
22 Y. Itoh, Chem. Rec., 2018, 18, 1681-1700.
23 G. Milman, D. M. Schwope, D. A. Gorelick and M. A. Huestis. Clin. Chim. Acta. 2012, 413, 765-770.
24 C. Coulter, M. Garnier and C. Moore, J. Anal. Toxicol., 2011, 35, 424-430.
25 Naqi, M. Tipping, C. R. Pudney S. M. Husbands and I. S. Blagbrough. 2019. Anal. Meth. 11, 3101-3107.

### SUPPORTING INFORMATION

Generally, where not included herein, Supporting Information. Parameters resulting from fitted FSFS. Parameters resulting from computational calculations. Photodegradation fingerprints for 1a-d. Analytical characterization from syntheses. This material is available free of charge via the Internet at http://pubs.acs.org.

### 3-(2-fluorobenzoyl)-1H-indole 1a

Sand-coloured solid (0.3354 g, 1.40 mmol, 32.8%); Mp 188.5-193.8° C.; ¹H NMR (500 MHZ, DMSO-d6) δ 12.13 (s, 1H), 8.24-8.16 (m, 1H), 7.79 (s, 1H), 7.61-7.56 (m, 2H), 7.54-7.50 (m, 1H), 7.38-7.31 (m, 2H), 7.30-7.23 (m, 2H). ¹³C NMR (126 MHz, DMSO-d6) δ186.1, 158.7 (d, J=247.4 Hz), 136.9, 131.8 (d, J=8.3 Hz), 129.7, 129.2, 129.1, 125.5, 124.4, 124.4, 123.3, 122.2, 121.1, 116.2 (d, J=14.7 Hz), 116.0, 112.4 ppm; ¹⁹F NMR (376 MHZ, DMSO-d6) δ −115.71 (dt, J=12.3, 7.1 Hz); IR (ATR) 3178.03 (N—H), 1613.49 (C═O) cm⁻¹; m/z: [M−H]⁻ Calculated for C₁₅H₁₀NOF 239.0746; Found 239.0740.

### 3-(2-chlorobenzoyl)-1H-indole 1b

Sand-coloured solid (0.5325 g, 2.08 mmol, 48.8%); Mp 178.1-182.8° C.; ¹H NMR (500 MHZ, DMSO-d₆) δ 12.11 (s, 1H), 8.16-8.12 (m, 1H), 7.64 (s, 1H), 7.59-7.44 (m, 5H), 7.30-7.21 (m, 2H) ppm; ¹³C NMR (126 MHZ, DMSO-d₆) δ 188.2, 140.3, 137.0, 136.9, 130.7, 129.7, 129.6, 128.7, 127.0, 125.4, 123.3, 122.2, 121.1, 116.0, 112.5 ppm; IR (ATR) 3226.72 (N—H), 1600.74 (C═O) cm⁻¹; m/z. [M−H]⁻ Calculated for C₁₅H₁₀NOCl 255.0451; Found 255.0446

### 3-(2-bromobenzoyl)-1H-indole 1c

Sand-coloured solid (0.8933 g, 2.98 mmol, 69.7%); Mp 174.6-177.0° C.; ¹H NMR (500 MHZ, DMSO-d₆) δ 12.11 (s, 1H), 8.13 (d, J=7.2 Hz, 1H), 7.73 (d, J=8.0 Hz, 1H), 7.62 (s, 1H), 7.53-7.47 (m, 3H), 7.46-7.42 (m, 1H), 7.26 (pd, J=7.1, 1.3 Hz, 2H) ppm; ¹³C NMR (126 MHZ, DMSO-d₆) δ 189.0, 142.3, 137.0, 136.9, 132.8, 130.8, 128.6, 127.5, 125.4, 123.3, 122.2, 121.1, 118.6, 115.7, 112.5 ppm; IR (ATR) 3150.78 (N—H), 1597.66 (C═O) cm⁻¹; m/z: [M−H]⁻ Calculated for C₁₅H₁₀NOBr 298.9946; Found 298.9938.

### 3-(2-iodobenzoyl)-1H-indole 1d

Sand-coloured solid (0.2876 g, 0.83 mmol, 19.4%); Mp 184.0-185.5° C.; ¹H NMR (500 MHZ, DMSO-d₆) δ 12.09 (s, 1H), 8.12 (d, J=7.1 Hz, 1H), 7.98-7.93 (m, 1H), 7.58 (d, J=2.0 Hz, 1H), 7.56-7.48 (m, 2H), 7.43 (dd, J=7.5, 1.6 Hz, 1H), 7.31-7.21 (m, 3H) ppm; ¹³C NMR (126 MHz, DMSO-d₆) δ 191.0, 146.1, 139.1, 137.0, 136.9, 130.7, 127.9, 127.8, 125.5, 123.3, 122.2, 121.2, 115.2, 112.5, 93.1 ppm; IR (ATR) 3146.12 (N—H), 1595.99 (C═O) cm⁻¹; m/z. [M−H]⁻ Calculated for C₁₅H₁₀NOI 346.9807; Found 346.9796.

### 2-(2-fluorophenyl)-1-(1H-indol-3-yl) ethanone 2a

Pale pink solid (0.3442 g, 1.35 mmol, 31.9%); Mp 194.5-195.0° C.; ¹H NMR (500 MHZ, DMSO-d₆) δ 12.01 (s, 1H), 8.49 (s, 1H), 8.15 (dd, J=7.8, 1.3 Hz, 1H), 7.49 (d, J=7.8 Hz, 1H), 7.40-7.26 (m, 2H), 7.26-7.12 (m, 4H), 4.28 (s, 2H); ¹³C NMR (126 MHz, DMSO-d₆) δ 191.03, 161.72, 159.78, 136.61, 134.25, 132.29, 132.25, 128.52, 128.46, 125.44, 124.11, 124.08, 123.47, 123.35, 122.84, 121.77, 121.21, 115.80, 115.00, 114.83, 112.13, 39.07; ¹⁹F NMR (376 MHz, DMSO-d₆) δ −116.97 (dt, J=10.5, 6.8 Hz); IR (ATR) 3176.25 (N—H), 1628.18 (C═O) cm⁻¹; m/z: [M−H]⁻ Calculated for C₁₆H₁₂NOF 253.0903; Found 253.0902.

### 2-(2-chlorophenyl)-1-(1H-indol-3-yl) ethanone 2b

Sand coloured solid (0.1114 g, 0.41 mmol, 9.62%); Mp 215.4-216.4° C.; ¹H NMR (500 MHZ, DMSO-d₆) δ 12.00 (s, 1H), 8.51 (s, 1H), 8.14 (d, J=7.8 Hz, 1H), 7.52-7.37 (m, 3H), 7.30 (qt, J=7.5, 3.8 Hz, 2H), 7.20 (dt, J=22.7, 7.1 Hz, 2H), 4.40 (s, 3H); ¹³C NMR (126 MHZ, DMSO-d₆) δ 190.92, 136.58, 134.52, 134.09, 133.81, 132.50, 128.90, 128.31, 126.91, 125.43, 122.82, 121.75, 121.22, 115.95, 112.13, 43.48; IR (ATR) 3214.02 (N—H), 1630.46 (C═O) cm⁻¹; m/z: [M−H]⁻ Calculated for C₁₆H₁₂NOCI 269.0607; Found 269.0607.

### 2-(2-bromophenyl)-1-(1H-indol-3-yl) ethanone 2c

Sand coloured solid (0.0950 g, 0.30 mmol, 7.04%); Mp 225.2-226.7° C.; ¹H NMR (500 MHZ, DMSO-d₆) δ 12.01 (s, 1H), 8.51 (s, 1H), 8.14 (d, J=7.6 Hz, 1H), 7.61 (dd, J=8.0, 1.0 Hz, 1H), 7.49 (d, J=8.0 Hz, 1H), 7.43-7.32 (m, 2H), 7.26-7.15 (m, 3H), 4.42 (s, 2H); ¹³C NMR (126 MHZ, DMSO-d₆) δ 190.85, 136.58, 136.31, 134.05, 132.55, 132.15, 128.51, 127.45, 125.43, 124.80, 122.80, 121.74, 121.22, 116.00, 112.13, 45.92; IR (ATR) 3213.34 (N—H), 1626.33 (C═O) cm⁻¹; m/z: [M−H]⁻ Calculated for C₁₆H₁₂NOBr 313.0102; Found 313.0101.

### 2-(2-iodophenyl)-1-(1H-indol-3-yl) ethanone 2d

Sand coloured solid (0.0780 g, 0.22 mmol, 5.07%); Mp 228.7-229.9° C.; ¹H NMR (500 MHZ, CDCl) δ 12.00 (s, 1H), 8.52 (d, J=3.1 Hz, 1H), 8.14 (d, J=7.7 Hz, 1H), 7.88-7.83 (m, 1H), 7.49 (d, J=8.1 Hz, 1H), 7.41-7.33 (m, 2H), 7.20 (dtd, J=22.1, 7.2, 1.2 Hz, 2H), 7.02 (ddd, J=8.0, 6.2, 2.9 Hz, 1H), 4.42 (s, 2H) ppm; ¹³C NMR (126 MHz, DMSO-d₆) δ 191.40, 140.35, 139.16, 137.05, 134.51, 132.07, 128.91, 128.55, 125.91, 123.27, 122.21, 121.72, 116.68, 112.60, 102.60, 50.79 ppm; IR (ATR) 3212.62 (N—H), 1629.81 (C═O) cm⁻¹; m/z. [M−H]⁻ Calculated for C₁₆H₁₂NOI 360.9964; Found 360.9964.

1. A drug detection method to identify whether a drug is present in a provided sample, the method comprising:
determining a first fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
determining a wavelength for irradiation of the provided sample to induce a photochemical change in the provided sample and irradiating the provided sample with radiation having the determined wavelength;
determining a second fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
comparing the determined first and second fluorescence spectral matrix associated with the provided sample with a library of first and second fluorescence spectral matrices obtained from a plurality of drug samples; and
if a match within a predetermined threshold is revealed by said comparison, triggering a positive identification of a drug in the provided sample.

2. A drug detection method according to claim 1, wherein determining a wavelength for irradiation of the provided sample to induce a photochemical change in the provided sample comprises: determining a wavelength of an absorption maxima of the provided sample and irradiating the provided sample with irradiation having a wavelength close to the determined absorption maxima to induce a photochemical change in the provided sample.

3. A drug detection method according to claim 1, wherein determining a wavelength for irradiation of the provided sample to induce a photochemical change in the provided sample comprises: measuring an absorption spectrum of a plurality of drugs and evaluating the measured absorption spectra to select one or more wavelength for irradiation of the provided sample.

4. A drug detection method according to claim 1, wherein the wavelength for irradiation comprises a wavelength in the ultraviolet region of the spectrum.

5. The drug detection method according to claim 1, wherein the drug comprises at least one of the following: a photochemically active aromatic molecule; or a molecule which photochemically changes to form an aromatic molecule; or a synthetic cannabinoid receptor agonist; or a cannabinoid; or an opioid; or a benzodiazepine; or a cathinone; or a steroid or a sedative.

6. The drug detection method according to claim 1, wherein the provided sample comprises: a drug in solution, and optionally wherein the provided sample comprises at least one of the following: a drug in ethanol solution; or a drug in saliva; or a combusted drug in saliva; or a drug in solid form.

7. The drug detection method according to claim 1, wherein the provided sample comprises: a drug adsorbed onto a physical matrix, and optionally wherein the physical matrix comprises paper.

8. The drug detection method according to claim 1, wherein triggering a positive identification of a drug comprises identifying which of the plurality of drug samples in the library is a source of a first and second fluorescence spectral matrix which both match said determined first and second fluorescence spectral matrix associated with the provided sample within the threshold.

9. The drug detection method according to claim 1, wherein triggering a positive identification of a drug comprises identifying which of the plurality of drug samples in the library is a source of a difference map between a first and second fluorescence spectral matrix which matches a difference map created by comparison of the determined first and second fluorescence spectral matrix associated with the provided sample within the threshold.

10. The drug detection method according to claim 1, wherein triggering a positive identification of a drug comprises identifying which of the plurality of drug samples in the library is a source of a difference map between a first and second fluorescence spectral matrix which matches a difference map created by comparison of the determined first and second fluorescence spectral matrix associated with the provided sample, and a first fluorescence spectral matrix which matches a determined first fluorescence spectral matrix associated with the provided sample within the threshold.

11. The drug detection method according to claim 1, wherein irradiating the provided sample comprises irradiating the provided sample for a predetermined time period at a selected intensity and optionally wherein the predetermined time period and intensity are selected to be likely to induce a photochemical change in a drug in the provided sample.

10. Drug detection apparatus configured to identify whether a drug is present in a provided sample, the apparatus comprising:
a controller, an excitation source and an emission detector;
the controller comprising at least one processor; and at least one memory storing instructions that, when executed by the at least one processor, cause the controller at least to perform:
determining a first fluorescence spectral matrix associated with the provided sample by:
causing excitation of the provided sample by the excitation source at a first excitation wavelength; and
receiving, from the emission detector, a fluorescence emission spectrum generated by the provided sample at the first excitation wavelength;
repeating the excitation and receiving steps across a range of excitation wavelengths which differ from the first excitation wavelength;
storing excitation and emission data obtained in each excitation and receiving step; and
communicating with the stored data to create the first fluorescence spectral matrix associated with the provided sample;
determining a wavelength for irradiation of the provided sample to induce a photochemical change in the provided sample and irradiating the provided sample with radiation having the determined wavelength;
determining a second fluorescence spectral matrix associated with the provided sample by:
causing excitation of the provided sample by the excitation source at a first excitation wavelength; and
receiving, from the emission detector, a fluorescence emission spectrum generated by the provided sample at the first excitation wavelength;
repeating the excitation and receiving steps across a range of excitation wavelengths which differ from the first excitation wavelength;
storing excitation and emission data obtained in each excitation and receiving step; and
communicating with the stored data to create the second fluorescence spectral matrix associated with the provided sample;
comparing the determined first and second fluorescence spectral matrix associated with the provided sample with a library of first and second fluorescence spectral matrices obtained from a plurality of drug samples; and
if a match within a predetermined threshold is revealed by said comparison, triggering a positive identification of a drug in the provided sample.

11. The drug detection apparatus according to claim 10, wherein determining a wavelength for irradiation of the provided sample to induce a photochemical change in the provided sample comprises: determining a wavelength of an absorption maxima of the provided sample and irradiating the provided sample with irradiation having a wavelength close to the determined absorption maxima to induce a photochemical change in the provided sample.

12. The drug detection apparatus according to claim 10, wherein determining a wavelength for irradiation of the provided sample to induce a photochemical change in the provided sample comprises: measuring an absorption spectrum of a plurality of drugs and evaluating the measured absorption spectra to select one or more wavelength for irradiation of the provided sample.

13. The drug detection apparatus according to claim 10, wherein the drug comprises at least one of the following: a photochemically active aromatic molecule; or a molecule which photochemically changes to form an aromatic molecule; or a synthetic cannabinoid receptor agonist; or a cannabinoid; or an opioid; or a benzodiazepine; or a cathinone; or a steroid or a sedative.

14. The drug detection apparatus according to claim 10, wherein the provided sample comprises: a drug in solution.

15. The drug detection apparatus according to claim 10, wherein said provided sample comprises at least one of the following: a drug in ethanol solution; or a drug in saliva; or a combusted drug in saliva; or a drug in solid form.

16. The drug detection apparatus according to claim 10, wherein the provided sample comprises: a drug adsorbed onto a physical matrix, and optionally wherein the physical matrix comprises paper.

17. The drug detection apparatus according to claim 10, wherein said excitation source comprises: an array of different LEDs selected to have an emission spectrum which, when taken together, spans a wavelength range from 250 nm to 400 nm.

18. The drug detection apparatus according to claim 10, wherein said controller is configured to control said array of different LEDs to selectively switch one or more LEDs in said array on or off and thereby control said first wavelength.

19. The drug detection apparatus according to claim 10, wherein the emission detector comprises a monochromated spectrometer.

20. The drug detection apparatus according to claim 10, wherein the library is stored on locally provided memory; and optionally wherein the apparatus comprises communication circuitry configured to communicate with a remotely stored library.

21. A non-transitory computer readable medium comprising program instructions stored thereon for performing a drug detection method to identify whether a drug is present in a provided sample, the instructions comprising at least the following:
determining a first fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
determining a wavelength for irradiation of the provided sample to induce a photochemical change in the provided sample and irradiating the provided sample with radiation having the determined wavelength;
determining a second fluorescence spectral matrix associated with the provided sample by:
excitation of the provided sample at a first excitation wavelength; and
receiving a fluorescence emission spectrum generated by the provided sample at the first excitation wavelength;
repeating the excitation and receiving steps across a range of excitation wavelengths which differ from the first excitation wavelength;
comparing the determined first and second fluorescence spectral matrix associated with the provided sample with a library of first and second fluorescence spectral matrices obtained from a plurality of drug samples; and
if a match within a predetermined threshold is revealed by said comparison, triggering a positive identification of a drug in the provided sample.
